(19) United States
(12) Patent Application Publication
(10) Pub. No.: US 2021/0123025 A1
(43) Pub. Date: Apr. 29, 2021

(54) CELLULAR AGGREGATES FOR USE IN VASCULARISATION THERAPY

(71) Applicants: UNIVERSIDAD DEL PAÍS VASCO - EUSKAL HERRIKO UNIBERTSITATEA, Leioa (ES); ACHUCARRO BASQUE CENTER FOR NEUROSCIENCE FUNDAZIOA, Leioa (ES)

(72) Inventors: José Ramón PINEDA MARTÍ, Leioa (ES); Jon LUZURIAGA GONZÁLEZ, Leioa (ES); Fernando UNDA RODRÍGUEZ, Leioa (ES); Oier PASTOR ALONSO, Leioa (ES); Juan Manuel ENCINAS PÉREZ, Leioa (ES); Gaskon IBARRETXE BILBAO, Leioa (ES); Igor IRASTORZA EPELDE, Leioa (ES)

(21) Appl. No.: 17/257,457

(22) PCT Filed: Jul. 2, 2019
(86) PCT No.: PCT/EP2019/067769

§ 371 Date: Dec. 31, 2020

(30) Foreign Application Priority Data
Jul. 3, 2018 (EP) 18382492.9

(51) Int. Cl. C12N 5/071; A61K 35/44; A61P 25/00; A61P 9/00

(52) U.S. Cl. CPC C12N 5/0691; A61K 35/44; A61P 25/00; A61P 9/00; C12N 2500/90; C12N 2506/1361; C12N 2501/11; C12N 2501/115; C12N 2501/91; C12N 2502/1364; C12N 2502/28; C12N 2501/165

(57) ABSTRACT

The present invention provides a serum-free endothelial cell differentiation culture medium comprising (a) a basal culture medium and (b) an endothelial cell differentiation combination of EGF, FGF and VEGF protein, wherein the amount of EGF is higher than the amount of FGF protein. The present invention further provides a process for the preparation of cellular aggregate suspensions comprising differentiated endothelial cells from dental stem cells using the serum-free medium, as well as the use of the resulting suspension in therapy.

This application claims the benefit of European Patent Application EP18382492.9 filed on Jul. 3, 2018.

### TECHNICAL FIELD

The present application generally relates to dental stem cell differentiation. More specifically, the invention is directed to a serum-free culture medium comprising epidermal growth factor (EGF) and fibroblast growth factor (FGF), which allows the differentiation of dental stem cells to endothelial cells. The methods provided by the present invention may involve the formation of dentospheres (i.e. cellular aggregates containing endothelial cells derived from dental stem cells). Accordingly, also methods for generating dentospheres are object of the present disclosure. The dentospheres of the invention show neovascularizing and neuroprotective effects, among others, making them useful in vascularization therapy, more particularly in neovascularization therapy.

### BACKGROUND ART

Human vascular endothelial cells are important for developing engineered vessels, for the treatment of vascular disease and may also be useful for augmenting vessel growth to areas of ischemic tissue or following implantation. Endothelial progenitor cells from adults have vasculogenic potential. This potential can be exploited in tissue engineering for induction of tissue vascularization, especially for complex tissues where vascularization of regenerating tissue is essential. For example, it is often desirable to vascularize engineered tissue in vitro prior to transplantation. Vascularization in vitro is important to enable cell viability during tissue growth, induce structural organization and promote integration upon implantation.

The use of stem cells in tissue engineering and other applications in place of adult endothelial progenitor or endothelial cells could be particularly exciting, since stem cells can be expanded without apparent limit and endothelial-derived cells could be created in virtually unlimited amounts and available for potential clinical use. A potential source of cells for these applications are dental stem cells (DSCs).

The dental pulp is a soft tissue of ectomesenchymal and mesenchymal origin, developing from the dental papilla. Stem cell populations can be isolated from different tissues of the oral and maxillofacial regions. They are obtained from different parts and developmental stages of the tooth. Around eight unique populations of dental tissue-derived mesenchymal stem cells (MSCs) have been isolated and characterized. Post-natal dental pulp stem cells (DPSCs) were the first human dental MSCs to be identified from pulp tissue. Other dental MSC-like populations, such as stem cells from human exfoliated deciduous teeth (SHED), periodontal ligament stem cells (PDLSCs), dental follicle progenitor cells (DFPCs), alveolar bone-derived mesenchymal stem cells (ABMSCs), stem cells from the apical part of the human dental papilla (SCAP), tooth germ progenitor cells (TGPCs), and gingival mesenchymal stem cells (GMSCs), were also isolated and characterized.

All the aforementioned dental cell populations share stemness properties and they are able to differentiate into several cell types, preferentially to those of mesenchymal lineages, like osteoblasts, chondroblasts and adipocytes.

DSCs have been widely studied due to their great clinical potential, easy accessibility, and less invasive harvesting. Several preclinical investigations conducted so far indicated the extensive potential of the stem cell in tissue repair and regeneration of dental tissues, as well as other organs.

In view of the above, efforts have been made in the development of new protocols to successfully achieve the differentiation of dental stem cells to endothelial cells. For example, it has been reported the ability of DPSCs to co-differentiate to endothelial cell phenotypes when cultured with Fetal Bovine Serum (FBS)-containing media. The possibility to use these cells as a source of young neovasculature has not been considered, despite the generation of a highly vascularized bone-like tissue after their subcutaneous graft in vivo. One of the limitations is the high presence of fetal serum (10-20% FBS). Fetal serum permits cell survival and rapid cell expansion for the subsequent application of differentiation protocols. However, its presence also facilitates the differentiation toward osteoblastic/odontoblastic lineages and might also cause allergies and immune reactions in the host.

The prior art has also reported that the differentiation to endothelial cell phenotype requires a step of adhesion of the stem cell to a scaffold/support (usually the Petri dish wherein the cultivation step occurs) and the subsequent isolation of the endothelial cells to use them in therapy. This means that the processes disclosed in the prior art require a further step to detach the differentiated cells from the scaffold/support, thus limiting the endothelial cell production at high scale for their use in therapy.

In spite of the efforts made, therefore, there is still the need of procedures which, taking profit of the easy bioavailability of dental stem cells, can provide high amounts of derivatized endothelial cells in an easy way.

### SUMMARY OF INVENTION

The present inventors have developed a process for obtaining endothelial marker expressing cells ready to use for in vivo grafts starting from genetically unmodified extracted human dental stem cells.

The invention is based on the finding that a combination of an amount of EGF with an amount of a FGF protein (FGF2) which is lower than the one of EGF, can be used in the endothelial cell differentiation of dental stem cells.

The present inventors have also developed a serum-free endothelial cell which, in addition to including EGF and FGF, includes VEGF. As it is shown below, the inclusion of VEGF in the serum-free medium improves the surprising endothelial differentiation effect of EGF+FGF (see Example 6, FIG. 6).

Thus, in a first aspect the present invention provides a serum-free endothelial cell differentiation culture medium comprising (a) a serum-free basal culture medium and (b) an endothelial cell differentiation combination comprising vascular endothelial growth factor (VEGF), EGF and a FGF protein, wherein the amount of EGF is higher than the amount of FGF.

Until the present invention, it was well-established in the state of the art that the culture medium needed the inclusion of serum, such as FBS, in order to support endothelial differentiation, cell survival and facilitate stem cell expansion. However, as mentioned above, the use of serum in endothelial differentiation has been reported as not convenient because the resulting product can give rise to undesirable side-effects in the host (such as immune side-reactions which lead to the rejection of cell transplants in humans). In addition, it has also been reported that the inclusion of serum in the culture medium gives rise to an adherent growth in a cell monolayer, which require further steps for using the cells in therapy.

As it is shown below, when the inventors prepared a suspension of isolated dental stem cells with a serum-free medium comprising EGF and FGF as defined in the first aspect of the invention, they found that there was an increase in the activity of the ERK and STAT3 signalling pathways, which had already been described in the state of the art to be essential to differentiate stem cells into endothelial cells. In the conditions described in the present invention, both signalling pathways are activated in DSCs. Further assays, wherein specific endothelial cell markers were analyzed (such as CD31 or VEGFR, among others) showed that upregulation of these markers in DPSCs can be abolished in the presence of inhibitors of STAT3 signalling (Stattic; at a concentration between 1 and 2.5 μM). Altogether, the above results allowed concluding to the inventors that the serum-free medium of the first aspect of the invention allowed the efficient differentiation to endothelial marker expressing cells, without the need of using scaffolds or serum-additions.

Thus, the present invention allows obtaining a safer and more functional product (the endothelial cell) to be used in humans.

On the other hand, it was also well-established that the use of a scaffold in endothelial differentiation limited the production of endothelial cells at large amounts as well as its scale-up in industrial plant.

The present inventors have found that floating cellular aggregates comprising dental stem cells as well as endothelial cells were obtained when the dental stem cells were cultured in suspension with the medium of the first aspect of the invention. That is, the particular properties of the culture medium of the first aspect of the invention allow the efficient endothelial differentiation of stem cells in the absence of a scaffold or support.

In addition, due to its particular properties, the culture medium of the invention can be further used in the proliferation/maintenance of the availability of the cells.

This means, therefore, a great advance in the efficient production of cultures with endothelial marker expressing cells in large amounts for their later use in therapy.

Thus, in a second aspect the present invention provides an in vitro serum-free process for the preparation of a cellular aggregate comprising dental stem cells and one or more differentiated endothelial cells, the process comprising culturing in suspension an isolated sample comprising dental stem cells in a serum-free cell culture medium which is selected from: (a) a free-serum culture medium comprising a basal culture medium, and an endothelial cell differentiation combination of EGF and a FGF protein, wherein the amount of EGF is higher than the amount of FGF protein; (b) a free-serum culture medium comprising a basal culture medium, EGF, FGF, heparin and a cell supplement, wherein the amount of EGF is higher than the amount of FGF protein; and (c) a free-serum culture medium as defined in the first aspect of the invention.

The present inventors have also found that the cellular aggregates resulting from the process of the second aspect of the invention have remarkable properties when compared to the aggregates obtained with a serum culture medium which does not include EGF and FGF protein: maintained expression of BDNF, enrichment in endothelial cells, expression of laminin and VEGFR2 in high amounts, it does not include serum components (due to the process), and has a strong vasculogenic and angiogenic effect.

In particular, the present inventors found that there was an increase of up to 26% of CD31-FITC positive cell population in dental stem cells when these cells were grown with the medium of the first aspect; contrary to the poor result achieved (0.3%) using the serum-containing culture medium. This means that the particular conditions of the process (absence of serum, absence of scaffold, and inclusion of an amount of EGF higher than the amount of FGF) substantially enrich the cellular aggregate in endothelial cells.

In addition to the above, the present inventors further found that dental stem cells cultured with the medium of the first aspect of the invention showed 17.9±10.2 fold-increase of VEGFR2 mRNA with respect to cultures with DMEM+10% FBS.

Therefore, in a third aspect, the present invention provides a cellular aggregate comprising dental stem cells and endothelial cells, which is obtainable by the process of the second aspect of the invention.

In a fourth aspect the present invention provides a culture, preferably a serum-free culture, comprising the cellular aggregate as defined in the third aspect of the invention.

In a fifth aspect the present invention provides the use of a combination comprising EGF and FGF, wherein the amount of EGF is higher than the amount of FGF, in the differentiation of an isolated stem cell, preferably a dental stem cell, more preferably a DPSC, to an endothelial cell.

In a sixth aspect the present invention provides the use of an endothelial cell differentiation culture medium comprising EGF and FGF, preferably the use of a serum-free endothelial cell differentiation culture medium, which is selected from:

(a) a free-serum culture medium comprising a basal culture medium, and an endothelial cell differentiation combination of EGF and a FGF protein, wherein the amount of EGF is higher than the amount of FGF protein; (b) a free-serum culture medium comprising a basal culture medium, EGF, FGF, heparin and a cell supplement, wherein the amount of EGF is higher than the amount of FGF protein; and (c) a free-serum culture medium as defined in the first aspect of the invention, in differentiating a stem cell, preferably a dental stem cell, to an endothelial cell.

In view of the in vitro results provided below, the cellular aggregates of the invention can be considered as potentially useful in autologous regenerative therapy.

Thus, in a seventh aspect the present invention provides a pharmaceutical composition comprising a therapeutically effective amount of the cellular aggregate as defined in the third aspect of the invention or the culture as defined in the fourth aspect together with pharmaceutically acceptable excipients or carriers.

In an eighth aspect the present invention provides a cellular aggregate as defined in the third aspect or the culture of the fourth aspect of the invention for use in therapy.

Without being bound to the theory, the inventors believe that, when the aggregate is applied, dental stem cells disaggregate and graft into the lesion (for example in brain, as shown below), and the CD31-positive endothelial cells can functionally integrate into vasculature, promoting de novo generation of new blood vessels within brain tissue. In this way the aggregate of the invention could promote the formation of the endothelial lining as well as of outer vascular structures, resulting in a robust blood vessel with a large lumen diameter, consistent with an arteriole/venule identity, as shown in FIG. 5.

As it is shown below, when the cellular aggregate of the third aspect of the invention was applied to a brain lesion in an immunosuppressed mice, it was found that the vasculature containing human cells presented histomorphological characteristics of healthy blood vessels, which expressed VEGF and had high levels of laminin staining, suggesting that grafted cells facilitated a process of angiogenesis and neovascularization.

In addition to the above, it was also found that the cellular aggregate of the invention maintained brain-derived neurotrophic factor (BNDF) expression, which has also been reported as potentially useful in vasculogenesis and neuroprotective therapies.

Therefore, in a ninth aspect the present invention provides the cellular aggregate as defined in the third aspect or the culture as defined in the fourth aspect or the pharmaceutical composition as defined in the seventh aspect of the invention for use in tissue regeneration.

In a tenth aspect the present invention provides the cellular aggregate as defined in the third aspect or the culture as defined in the fourth aspect or the pharmaceutical composition as defined in the seventh aspect of the invention for use as angiogenic, vasculogenic and/or neuroprotective agent.

In connection with the above, it has also been reported that laminin is able to induce depolymerization of amyloid fibrils, reducing the toxic effects of amyloid peptides. Therefore, from a point of view of biological or therapeutic interest, the increase of vascular laminin observed in blood vessels containing dental stem cells-derived cells of the invention can also have beneficial effects in the case of neurodegenerative illnesses such as Alzheimer's disease.

Thus, in an eleventh aspect the present invention provides a cellular aggregate as defined in the third aspect of the invention or the culture as defined in the fourth aspect or the pharmaceutical composition as defined in the seventh aspect of the invention for use in the treatment or prevention of a disease caused by a reduction in the amount of laminin. This aspect can alternatively be formulated as the use of the cellular aggregate of the third aspect or the culture of the fourth aspect of the invention for the manufacture of a medicament for the treatment or prevention of a disease caused by a reduction in the amount of laminin. This aspect can alternatively be formulated as a method for the treatment or prevention of a disease caused by a reduction in the amount of laminin, the method comprising administering an effective amount of the cellular aggregate as defined in the third aspect of the invention or of the culture as defined in the four aspect or of the composition as defined in the seventh aspect of the invention, to a subject in need thereof.

Finally, a potential rejuvenation-like beneficial effect can also occurred due to laminin increase production because, as it has been reported in the prior art, one of the main extracellular matrix components that progressively decreases up to 50% during ageing is laminin.

### BRIEF DESCRIPTION OF DRAWINGS

FIG. 1: A) (left) Human DPSCs seeded in standard culture medium A acquire a flat morphology and adhere to the flask surface. However, when cultured with medium B (middle) DPSCs start to grow forming free-floating dentospheres. (right) Control neurospheres of murine NSCs. B) Population Doubling rates for human DPSCs grown either with medium A or medium B compared with murine NSCs. C) Cumulative population doubling (CPD) of DPSCs seeded in standard culture medium A or medium B and NSCs cultured in medium B. All different cell culture conditions were assessed in parallel (n=3 for each) for a total of 4 passages and non-parametric Kruskal-Wallis with post-hoc test was used (Mean±SD *p<0.05, **p<0.01 and ***p<0.001). Scale bar 75 μm.

FIG. 2: A) mRNA fold change of the endothelial marker CD31 on DPSC cultures depending on the culture medium: medium A vs. medium B. Axis represented as logarithmic scale of CD31 expression from DPSC cultures of 4 different patients (Mean±SEM *p=0.05, one-tailed Mann Whitney test). B) Quantification of the immunolabeling signal intensity for CD31 (Mean±SEM n=10 for medium A and n=10 for medium B, ***p=0.0008, one-tailed Mann Whitney test), and C) Flow cytometry analysis for CD31 expression in DPSC cultures (n=10.000 events for each condition. *p=0.05, one-tailed Mann Whitney test).

FIG. 3: A) RT-PCR detection for VEGF, confirming its expression in both medium A and medium B-grown DPSCs. A′) mRNA fold change of the endothelial marker VEGFR2 on DPSC cultures depending upon the type of media: medium A vs. medium B. Axis represented as logarithmic scale (Mean±SEM of three independent samples, ***p=0.0002, one-tailed, Mann Whitney test. B) Quantification of distribution of the average quantity of bright pixels per cell corresponding to VEGFR2 staining (n=33 cells for medium A and n=69 cells for medium B. ***p=0.0001, one tailed, Mann Whitney test). C) Quantification of distribution of the average of bright pixels per cell corresponding to phospho-ERK1/2 staining (n=30 cells for medium A and n=47 cells for medium B. ***p=0.0001, one tailed, Mann Whitney test).

FIG. 4: Western blot membranes of 200.000 cells for each condition showing A) phospho-ERK1/2 and total ERK protein levels and B) phospho-STAT3 and total STAT3 for DPSCs grown either with medium A (D) or medium B (N) culture media. As a positive control, human liver sinusoidal endothelial cells (L, LSEC) were used. A′) Quantification of phospho-ERK1/2 western blot in three different samples for each (D, N, L) condition (Mean±SEM; *p=0.04 (D vs N) and *p=0.0273 (D vs L); one-tailed Kruskal-Wallis test). B′) Quantification of phospho-STAT3 (Mean±SEM; *p=0.05 (D vs N); one-tailed Kruskal-Wallis test).

FIG. 5: One month post-graft into the hippocampus of Athymic nude mice, human cells integrate into murine vasculature. A) (left) Detail of human Nestin-positive DPSC-derived cells covering brain vasculature showed an increase in the staining for Laminin (middle) and VEGF (right), suggesting de novo vasculature formation, and its functionality as assessed by VEGF production. B) (left) CD31 staining for both murine and human endothelial cells colocalizing with human Nestin-positive DPSC-derived cells in some blood vessels (right), showing that all endothelial cells within the blood vessel are actually of human origin. C) A specific antibody for human-CD31 cells recognizes human DPSC-derived endothelial cells in blood vessels (left), and those vessels precisely correspond to the ones showing an increased laminin staining (middle). The merge of the images (right) shows no overlap of channels. C′) Quantification of laminin staining intensity for murine blood vessels and blood vessels containing human DPSC-derived endothelial cells (Mean±SEM n=30 and 35 blood vessels, respectively for n=4 grafted animals, ***p=0.0001, one-tailed Mann Whitney test), Scale bar 20 μm. D) 3D-reconstruction from a 10 μm thick cryostat slice showing human-CD31+ endothelial cells (IN GREEN, inner bright non-nuclear flat cell staining) in the inner wall of a medium size blood-vessel (venule) decorated with laminin staining (IN RED, darkest staining). Scale bar 5 μm. D′) Close detail of a human-CD31 positive cell showing the elongated nuclear morphology characteristic of endothelial cells (Arrow). The nuclei of all the cells forming the blood vessel (DAPI stain), including both endothelial and mural cells, are shown in grayscale. Scale bar 5 μm.

FIG. 6: A) Dissociated human DPSCs seeded in serum-free culture media B, C or D start to grow forming free-floating dentospheres already visible at 7 days in vitro. B) Immunolabeling signal intensity for CD31 of dissociated cells cultured on laminin-coated slices using medium C at 10 days in vitro. C) Immunolabeling signal intensity for CD31 of dissociated cells cultured on laminin-coated slices using medium D. Scale bar 50 μm, DIV=days in vitro.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition. The definitions given herein are included for the purpose of understanding and expected to be applied throughout description, claims and drawings.

The present invention provides in a first aspect a serum-free endothelial cell differentiation culture medium comprising VEGF, EGF and a FGF protein.

The term “serum-free”, when referred to the medium of the first aspect of the invention, means that it does not include serum or serum-derived compositions comprising seric antigenic components which can give rise to immune system side-effect reaction.

Throughout the specification the term “differentiated endothelial cell” refers to any cell resulting from the differentiation of a stem cell which (a) expresses one or more endothelial markers, and (b) has the same genetic load as the starting stem cell. In one embodiment, the endothelial cell expresses one or more of the following markers: CD31, VEGFR2, CD34, CD45, ICAM-1/CD54, LYVE-1, Tie-2/TEK, VCAM-1/CD106, VE cadherin and von Willebrand factor. In another embodiment, the endothelial cell expresses at least the endothelial markers CD31 and VEGFR2.

The determination of the markers expressed in the differentiated endothelial cell can be performed using well-known protocols based on nucleic acid amplification such as PCR or Q-PCR. Illustrative non-limitative examples of the primers and conditions that can be used for determining the expression of CD31 and VEGFR2 are provided in Table 1 below.

The genetic load can be determined following well-routine sequencing methods.

As mentioned above, the culture medium object of the present invention comprises a basal culture medium together with VEGF, EGF and FGF.

In the present invention, the term “serum-free basal culture medium” (also known as undefined medium or complex medium) refers to a serum-free culture medium containing all the elements that most cells need for growth and are not selective, so they are used for the general cultivation and maintenance of cells kept in laboratory culture collections. The basal medium contains: a carbon source such as glucose, water, salts, and a source of amino acids and nitrogen (e.g., beef, yeast extract). There is a high number of basal culture medium available in the state of the art. Illustrative non-limitative examples of basal culture mediums are MEM, α-MEM, DMEM, DMEM/F12, RPMI, or M3, among others.

Epidermal growth factor (“EGF”) stimulates cell growth and differentiation by binding to its receptor, EGFR. Human EGF is a 6-kDa protein with 53 amino acid residues and three intramolecular disulphide bonds. EGF protein is also commercially available and its sequence is well-known in the state of the art. For example for the human EGF the UniProt number P01133 (Version 2 of Jul. 7, 2009). “EGF” also embraces any of the isoforms of the sequence. “EGF” is the founding member of the EGF-family of proteins. Members of this protein family have highly similar structural and functional characteristics. Thus, the term “EGF” also encompasses these EGF-family proteins. Illustrative non-limitative examples of these other family members include: Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4). In one embodiment of the first aspect of the invention, optionally in combination with any of the embodiments provided above or below, the EGF protein is the canonical EGF protein. In another embodiment of the first aspect of the invention, optionally in combination with any of the embodiments provided above or below, the EGF protein is the canonical human EGF protein.

In another embodiment, optionally in combination with any of the embodiments provided above or below, the EGF is present in the culture medium of the first aspect of the invention in an amount from 1 ng to 100 ng, from 5 to 50 ng, or from 10 to 30 ng per mL of serum-free medium. In another embodiment, optionally in combination with any of the embodiments provided above or below, the EGF is present in the culture medium of the first aspect of the invention in an amount of 10, 11, 12, 13, 14, 15, 16, 17, 18, 19, 20, 21, 22, 23, 24, 25, 26, 27, 28, 29 or 30 ng per mL of serum-free medium. In another embodiment, optionally in combination with any of the embodiments provided above or below, the EGF is present in the culture medium of the first aspect of the invention in an amount of 20 ng per mL of serum-free medium.

Fibroblast growth factor (“FGF”) designates a family of cell signalling proteins that are involved in a wide variety of processes, most notably as crucial elements for normal development. Any irregularities in their function lead to a range of developmental defects. These growth factors generally act as systemic or locally circulating, extracellular signalling molecules that activate cell surface receptors, but a defining property of FGFs is that they bind to heparin and heparan sulphate thus some of them are found to be sequestered in the extracellular matrix of tissues that contains heparan sulphate proteoglycans and they are released locally upon injury or tissue remodelling. This family is formed by 22 members, all of which are structurally related signalling molecules. Members FGF1 through FGF10 all bind fibroblast growth factor receptors (FGFRs). FGF1 is also known as acidic fibroblast growth factor, and FGF2 is also known as basic fibroblast growth factor. In one embodiment of the first aspect of the invention, optionally in combination with any of the embodiments provided above or below, the FGF protein is FGF2.

FGF2, also known as basic fibroblast growth factor (bFGF) or FGF-6, is a growth factor and signalling protein encoded by the FGF2 gene. It is synthesized primarily as a 155 amino acid polypeptide, resulting in an 18 kDa protein. Like other FGF family members, basic fibroblast growth factor possess broad mitogenic and cell survival activities, and is involved in a variety of biological processes, including embryonic development, cell growth, morphogenesis, tissue repair, tumour growth and invasion. And up to now FGF2 had been reported as a critical component for the stem cells to remain in an undifferentiated state. FGF2 sequence is available in several databases such as Uniprot (for example the human FGF2 has the Uniprot accession number P09038, Version 3, Oct. 13, 2009) and it is also commercially available. In one embodiment of the first aspect of the invention, the FGF protein corresponds to the human FGF-2 protein.

In another embodiment, optionally in combination with any of the embodiments provided above or below, the FGF protein is present in the culture medium of the first aspect of the invention in an amount from 1 ng to 100 ng, from 5 to 50 ng, or from 8 to 20 ng per mL of serum-free medium. In another embodiment, optionally in combination with any of the embodiments provided above or below, the FGF protein is present in the culture medium of the first aspect of the invention in an amount of 8, 9, 10, 11, 12, 13, 14, 15, 16, 17, 18, 19 or 20 ng per mL of serum-free medium. In another embodiment, optionally in combination with any of the embodiments provided above or below, the FGF is present in the culture medium of the first aspect of the invention in an amount of 10 ng per mL of serum-free medium.

Vascular Endothelial Growth Factor (“VEGF”) is a 19.3-kDa monomer or 38.6-kDa dimer heparin-binding glycoprotein involved in vasculogenesis and angiogenesis. Proximal splice-site selection in exon 8 results in pro-angiogenic VEGFxxx isoforms (xxx is the number of aminoacids). VEGF promotes growth and survival of vascular endothelial cells. VEGF protein is commercially available and its sequence is well-known in the state of the art. For example for the human VEGF the UniProt number P15692 (version 16 of November 2001). “VEGF” also embraces any of the isoforms of the sequence. “VEGF” is the founding member of the VEGF-family of proteins. Members of this protein family have highly similar structural and functional characteristics. Thus, the term “VEGF” also encompasses these VEGF-family proteins. Illustrative non-limitative examples of these other family members include: vascular permeability factor (VPF), vascular endothelial growth factor A (“VEGF-A”) isoforms/variants: a, b, c, d, e, f, g, h, r, s. VEGF-B, VEGF-C and VEGF-D and placenta growth factor (PGF). In another embodiment of the first aspect of the invention, optionally in combination with any of the embodiments provided above or below, the VEGF protein is the canonical human VEGF protein. In another embodiment of the first aspect of the invention, optionally in combination with any of the embodiments provided above or below the VEGF glycoprotein corresponds to the human recombinant VEGF-165 glycoprotein.

In another embodiment, optionally in combination with any of the embodiments provided above or below, the VEGF is present in the culture medium of the first aspect of the invention in an amount from 1 ng to 100 ng per mL of serum-free medium. In another embodiment, optionally in combination with any of the embodiments provided above or below, the VEGF is present in the culture medium of the first aspect of the invention in an amount of 10, 15, 20, 25, 30, 35, 40, 45, 50, 55, 60, 65, 70, 75, 80, 85, 90, 95 or 100 ng per mL of serum-free medium. In another embodiment of the first aspect of the invention, optionally in combination with any of the embodiments provided above or below, the VEGF glycoprotein corresponds to the human recombinant VEGF-165 glycoprotein and it is present in an amount from 1 ng to 100 ng per mL of serum-free medium. In another embodiment of the first aspect of the invention, optionally in combination with any of the embodiments provided above or below, the VEGF glycoprotein corresponds to the human recombinant VEGF-165 glycoprotein and it is in an amount of 10, 15, 20, 25, 30, 35, 40, 45, 50, 55, 60, 65, 70, 75, 80, 85, 90, 95 or 100 ng per mL of serum-free medium.

In another embodiment of the first aspect of the invention, optionally in combination with any of the embodiments provided above or below, the medium comprises the canonical human EGF protein as well as the human FGF2 as endothelial cell differentiation factors.

In still another embodiment of the first aspect of the invention, optionally in combination with any of the embodiments provided above or below, the weight ratio EGF:FGF protein is higher than 1.1:1. In another embodiment of the first aspect of the invention, optionally in combination with any of the embodiments provided above or below, the weight ratio EGF:FGF protein is comprised from 1.1:1 to 20:1 or from 1.5:1 to 8:1. In another embodiment of the first aspect of the invention, optionally in combination with any of the embodiments provided above or below, the weight ratio EGF:FGF protein is 1.1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1. In another embodiment of the first aspect of the invention, optionally in combination with any of the embodiments provided above or below, the weight ratio EGF:FGF protein is 2:1.

In another embodiment of the first aspect of the invention, optionally in combination with any of the embodiments provided above or below, VEGF is in a weight ratio (i.e., the relation of weights) from 0.1 to an excess with respect to FGF. In another embodiment of the first aspect of the invention, optionally in combination with any of the embodiments provided above or below, FGF is in a weight ratio with respect to VEGF (i.e., the relation of weights) from 0.1:1 to 1:20, particularly from 0.5:1 to 1:15. In another embodiment of the first aspect of the invention, optionally in combination with any of the embodiments provided above or below, the weight ratio FGF: VEGF is from 1:1 to 1:10. In another embodiment of the first aspect of the invention, optionally in combination with any of the embodiments provided above or below, VEGF is in a weight ratio excess with respect to FGF.

In still another embodiment of the first aspect of the invention, optionally in combination with any of the embodiments provided above or below, the weight ratio EGF:VEGF:FGF is comprised from 1:1.1:1 to 15:30:1 or from 1:1.5:1 to 10:40:1. In another embodiment of the first aspect of the invention, optionally in combination with any of the embodiments provided above or below, the weight ratio EGF:VEGF:FGF protein is 1:2:1, 10:20:1.

In another embodiment of the first aspect of the invention, optionally in combination with any of the embodiments provided above or below, the serum-free medium of the first aspect of the invention comprises
(a) a serum-free basal medium, and
(b) EGF in an amount from 10 to 30 ng per mL of serum-free medium, and the weight ratio EGF:FGF protein is comprised from 1.5:1 to 8:1.

In another embodiment of the first aspect of the invention, optionally in combination with any of the embodiments provided above or below, the serum-free medium of the first aspect of the invention comprises
(a) a serum-free basal medium, and
(b) EGF and FGF protein at a weight ratio 2:1.

In another embodiment of the first aspect of the invention, optionally in combination with any of the embodiments provided above or below, the serum-free medium of the first aspect of the invention comprises
(a) a serum-free basal medium, and
(b) EGF in an amount of 20 ng per mL of serum-free medium with FGF protein in an amount of 10 ng per mL of serum-free medium.

In another embodiment of the first aspect of the invention, optionally in combination with any of the embodiments provided above or below, the serum-free medium of the first aspect of the invention comprises
(a) a serum-free basal medium, and
(b) VEGF in an amount from 10 to100 ng per mL of serum-free medium, and the weight ratio EGF:VEGF:FGF protein is comprised from 1:1.1:1 to 15:30:1.

In another embodiment of the first aspect of the invention, optionally in combination with any of the embodiments provided above or below, the serum-free medium of the first aspect of the invention comprises
(a) a serum-free basal medium, and
(b) EGF:VEGF:FGF protein at a weight ratio 1:2:1.

In another embodiment of the first aspect of the invention, optionally in combination with any of the embodiments provided above or below, the serum-free medium of the first aspect of the invention comprises
(a) a serum-free basal medium, and
(b) EGF:VEGF:FGF protein at a weight ratio 10:20:1.

In another embodiment of the first aspect of the invention, optionally in combination with any of the embodiments provided above or below, the serum-free endothelial cell differentiation culture medium further comprises one or more of heparin and a serum-free cell supplement.

Heparin is a naturally occurring anticoagulant produced by basophils and mast cells. In therapeutic doses, it acts as an anticoagulant, preventing the formation of clots and extension of existing clots within the blood. In cell cultures, heparin is added to stabilize FGF protein and in the other hand, to act as a chaperone, a substrate modulator, that enhances the susceptibility of the EGFR to phosphorylate after presence of EGF. It is commercially available and it is widely used in the growth of adult (non-embrionic) cells.

In another embodiment of the first aspect of the invention, optionally in combination with any of the embodiments provided above or below, heparin is in an amount from 1 μg to 20 μg per mL of serum-free culture medium. In another embodiment of the first aspect of the invention, optionally in combination with any of the embodiments provided above or below, the heparin is in an amount from 1 to 10 μg per mL of serum-free medium. In another embodiment of the first aspect of the invention, optionally in combination with any of the embodiments provided above or below, the heparin is in an amount of 1, 2, 3, 4, 5, 6, 7, 8, 9, or 10 μg per mL of serum-free medium. In another embodiment of the first aspect of the invention, optionally in combination with any of the embodiments provided above or below, the heparin is in an amount of 2 μg per mL of serum-free medium. In another embodiment of the first aspect of the invention, optionally in combination with any of the embodiments provided above or below, heparin is at a weight ratio in excess with respect to VEGF. In another embodiment of the first aspect of the invention, optionally in combination with any of the embodiments provided above or below, VEGF is at a weight ratio with respect to heparin comprised from 1:2 to 1:1000. In another embodiment of the first aspect of the invention, optionally in combination with any of the embodiments provided above or below, VEGF is at a weight ratio vs heparin from 1:10 to 1:700. In another embodiment of the first aspect of the invention, optionally in combination with any of the embodiments provided above or below, VEGF is at a weight ratio vs heparin from 1:30 to 1:500. In another embodiment of the first aspect of the invention, optionally in combination with any of the embodiments provided above or below, VEGF is at a weight ratio vs heparin is from 1:30 to 1:50 to 1:400 to 1:500. In another embodiment of the first aspect of the invention, optionally in combination with any of the embodiments provided above or below, VEGF is at a weight ratio vs heparin around 1:48 or 1:480.

In the present invention, the term “weight ratio” refers to the relation of weights of heparin vs VEGF (heparin:VEGF).

In another embodiment of the first aspect of the invention, optionally in combination with any of the embodiments provided above or below, heparin is at a weight ratio with respect to EGF in excess. In another embodiment of the first aspect of the invention, optionally in combination with any of the embodiments provided above or below, heparin is at a weight ratio excess with respect to EGF comprised from 80:1 to 120:1. In another embodiment of the first aspect of the invention, optionally in combination with any of the embodiments provided above or below, EGF is at a weight ratio with respect to heparin comprised from 1:90 to 1:110. In another embodiment of the first aspect of the invention, optionally in combination with any of the embodiments provided above or below, EGF is at a weight ratio with respect to heparin of 1:90, 1:91, 1:92, 1:93, 1:94, 1:95, 1:96, 1:97, 1:98, 1:99, 1:100, 1:101, 1:102, 1:103, 1:104, 1:105, 1:106, 1:107, 1:108, 1:109, or 1:110. In another embodiment of the first aspect of the invention, optionally in combination with any of the embodiments provided above or below, EGF is at a weight ratio with respect to heparin comprised of 1:100. In another embodiment of the first aspect of the invention, optionally in combination with any of the embodiments provided above or below, EGF is at a weight ratio excess with respect to heparin comprised from 1:2 to 1:1000. In another embodiment of the first aspect of the invention, optionally in combination with any of the embodiments provided above or below, EGF is at a weight ratio with respect to heparin comprised from 1:10 to 1:500. In another embodiment of the first aspect of the invention, optionally in combination with any of the embodiments provided above or below, EGF is at a weight ratio with respect to heparin from 1:100 to 1:300. In another embodiment of the first aspect of the invention, optionally in combination with any of the embodiments provided above or below, EGF is at a weight ratio with respect to heparin from 1:150 to 1:250. In another embodiment of the first aspect of the invention, optionally in combination with any of the embodiments provided above or below, EGF is at a weight ratio with respect to heparin of 1:240.

In the present invention, the term “weight ratio” refers to the relation of weights of EGF vs FGF protein (EGF:FGF) or of heparin vs EGF (heparin:EGF).

In the present invention, by “cell supplement” is understood any composition useful to support cell culture and which comprises, at least, vitamins (biotin, vitamin B12, and vitamin E (tocopherol)), hormones (corticosterone and progresterone), and antioxidants (glutathione, and superoxide dismutase). Further components that can be included are transferrin, selenium, and L-carnitine. In one embodiment the cell supplement lacks vitamin A. In another embodiment, the cell supplement is B-27, preferably lacking vitamin A.

The skilled person in the art knows that the B-27 is a serum-free supplement for the cultivation of and long-term viability of embryonic, post-natal and adult neurons. (for more information and details Chen Y. et al, 2008; Brewer G. J. et al., 1989; Romijn H. J., 1988; and Romijn H. J. et al., 1984).

In one embodiment of the first aspect of the invention, optionally in combination with any of the embodiments provided above or below, the cell supplement, preferably the B-27 supplement, preferably the B-27 supplement lacking vitamin A, is added to the basal medium at a volume ratio from 1:1 to basal medium excess. In one embodiment of the first aspect of the invention, optionally in combination with any of the embodiments provided above or below, the cell supplement, preferably the B-27 supplement, preferably the B-27 supplement lacking vitamin A, is added to the basal medium at a volume ratio from 1:25 to 1:200 or from 1:30 to 1:70. In one embodiment of the first aspect of the invention, optionally in combination with any of the embodiments provided above or below, the cell supplement, preferably the B-27 supplement, preferably the B-27 supplement lacking vitamin A, is added to the basal medium at a volume ratio of 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60 or 1:100. In one embodiment of the first aspect of the invention, optionally in combination with any of the embodiments provided above or below, the cell supplement, preferably the B-27 supplement, preferably the B-27 supplement lacking vitamin A, is added to the basal medium at a volume ratio of 1:50.

In the present invention the term “volume ratio” means the volume ratio between the cell supplement and the basal medium.

In one embodiment of the first aspect of the invention, optionally in combination with any of the embodiments provided above or below, the culture medium of the first aspect of the invention comprises the basal medium together with EGF, FGF protein, and the cell supplement, preferably the B-27, preferably B-27 supplement lacking vitamin A. In another embodiment of the first aspect of the invention, optionally in combination with any of the embodiments provided above or below, the culture medium of the first aspect of the invention comprises the basal medium together with canonical EGF, FGF2 and the cell supplement, preferably the B-27, preferably B-27 supplement lacking vitamin A. In one embodiment of the first aspect of the invention, optionally in combination with any of the embodiments provided above or below, the culture medium of the first aspect of the invention comprises the basal medium together with VEGF, EGF, FGF protein, and the cell supplement, preferably the B-27, preferably B-27 supplement lacking vitamin A. In another embodiment of the first aspect of the invention, optionally in combination with any of the embodiments provided above or below, the culture medium of the first aspect of the invention comprises the basal medium together with VEGF-165, EGF, FGF and the cell supplement, preferably the B-27, preferably B-27 supplement lacking vitamin A. In another embodiment of the first aspect of the invention, optionally in combination with any of the embodiments provided above or below, the culture medium of the first aspect of the invention comprises the basal medium together with VEGF, canonical EGF, FGF2 and the cell supplement, preferably the B-27, preferably B-27 supplement lacking vitamin A. In another embodiment of the first aspect of the invention, optionally in combination with any of the embodiments provided above or below, the culture medium of the first aspect of the invention comprises the basal medium together with VEGF-165, canonical EGF, FGF2 and the cell supplement, preferably the B-27, preferably B-27 supplement lacking vitamin A. In another embodiment of the first aspect of the invention, optionally in combination with any of the embodiments provided above or below, the culture medium of the first aspect of the invention comprises the basal medium together with EGF, FGF protein, and heparin. In another embodiment of the first aspect of the invention, optionally in combination with any of the embodiments provided above or below, the culture medium of the first aspect of the invention comprises the basal medium together with VEGF, EGF, FGF protein, and heparin. In another embodiment of the first aspect of the invention, optionally in combination with any of the embodiments provided above or below, the culture medium of the first aspect of the invention comprises the basal medium together with VEGF-165, EGF, FGF protein, and heparin. In another embodiment of the first aspect of the invention, optionally in combination with any of the embodiments provided above or below, the culture medium of the first aspect of the invention comprises the basal medium together with canonical EGF, FGF2 and heparin. In another embodiment of the first aspect of the invention, optionally in combination with any of the embodiments provided above or below, the culture medium of the first aspect of the invention comprises the basal medium together with VEGF, canonical EGF, FGF2 and heparin. In another embodiment of the first aspect of the invention, optionally in combination with any of the embodiments provided above or below, the culture medium of the first aspect of the invention comprises the basal medium together with VEGF-165, canonical EGF, FGF2 and heparin. In another embodiment of the first aspect of the invention, optionally in combination with any of the embodiments provided above or below, the culture medium of the first aspect of the invention comprises the basal medium together with EGF, FGF protein, heparin, and the cell supplement, preferably the B-27 supplement, preferably B-27 supplement lacking vitamin A. In another embodiment of the first aspect of the invention, optionally in combination with any of the embodiments provided above or below, the culture medium of the first aspect of the invention comprises the basal medium together with VEGF, EGF, FGF protein, heparin, and the cell supplement, preferably the B-27 supplement, preferably B-27 supplement lacking vitamin A. In another embodiment of the first aspect of the invention, optionally in combination with any of the embodiments provided above or below, the culture medium of the first aspect of the invention comprises the basal medium together with VEGF-165, EGF, FGF protein, heparin, and the cell supplement, preferably the B-27 supplement, preferably B-27 supplement lacking vitamin A. In another embodiment of the first aspect of the invention, optionally in combination with any of the embodiments provided above or below, the culture medium of the first aspect of the invention comprises the basal medium together with canonical EGF, FGF2, heparin and a cell supplement, preferably the B-27 supplement, preferably B-27 supplement lacking vitamin A. In another embodiment of the first aspect of the invention, optionally in combination with any of the embodiments provided above or below, the culture medium of the first aspect of the invention comprises the basal medium together with VEGF, canonical EGF, FGF2, heparin and a cell supplement, preferably the B-27 supplement, preferably B-27 supplement lacking vitamin A. In another embodiment of the first aspect of the invention, optionally in combination with any of the embodiments provided above or below, the culture medium of the first aspect of the invention comprises the basal medium together with VEGF-165, EGF, FGF protein, heparin, and the cell supplement, preferably the B-27 supplement, preferably B-27 supplement lacking vitamin A.

In another embodiment of the first aspect of the invention, optionally in combination with any of the embodiments provided above or below, the serum-free medium of the first aspect of the invention comprises
(a) a serum-free basal medium, and
(b) EFG and FGF protein at a weight ratio EGF:FGF protein comprised from 1.5:1 to 8:1; heparin in an amount between 1 μg and 4 μg per mL of the culture medium; and the cell supplement, preferably the B-27 supplement, preferably B-27 supplement lacking vitamin A, at a volume ratio vs basal medium comprised from 1:25 to 1:200.

In another embodiment of the first aspect of the invention, optionally in combination with any of the embodiments provided above or below, the serum-free medium of the first aspect of the invention comprises
(a) a serum-free basal medium, and
(b) EGF in an amount from 10 to 30 ng, the weight ratio EGF:FGF protein being comprised from 1.5:1 to 8:1; heparin in an amount from 1 to 100 μg per mL of culture medium; and the cell supplement, preferably the B-27 supplement, preferably the B-27 supplement lacking vitamin A, at a volume ratio vs basal medium comprised from 1:25 to 1:200.

In another embodiment of the first aspect of the invention, optionally in combination with any of the embodiments provided above or below, the serum-free medium of the first aspect of the invention comprises
(a) a serum-free basal medium, and
(b) EGF and FGF protein at a weight ratio 2:1, heparin in an amount between 1 μg and 4 μg per mL of culture medium; and a cell supplement, preferably the B-27 supplement, preferably the B-27 supplement lacking vitamin A, at a volume ratio vs the basal medium of 1:10 to 1:100.

In another embodiment of the first aspect of the invention, optionally in combination with any of the embodiments provided above or below, the serum-free medium of the first aspect of the invention comprises
(a) a serum-free basal medium, and
(b) EGF in an amount of 20 ng, EGF:FGF protein weight ratio being 2:1; heparin in an amount of 2 μg/mL per mL of culture medium; and a cell supplement, preferably the B-27 supplement, preferably the B-27 supplement lacking vitamin A, at a volume ratio vs the basal medium of 1:50.

The culture of the invention can include other components which can help in endothelial proliferation. For example, the medium may comprise a solution of N2, other endothelial differentiation induced factors, commercially endothelial cells, antibiotics, such as penicillin and streptomycin, and other typical agents such as glutamate, glutamax, pyruvate, fungizone, glucose, zinc, selenite and cAMP.

In one embodiment, optionally in combination with any of the embodiments provided above or below, the serum-free medium of the first aspect of the invention further includes VEGF.

In another embodiment of the first aspect of the invention, optionally in combination with any of the embodiments provided above or below, VEGF is in an amount from 1 to 100 ng per mL of serum-free medium. In another embodiment of the first aspect of the invention, optionally in combination with any of the embodiments provided above or below, VEGF is in an amount of 1, 10, 20, 30, 40, 50, 60, 70, 80, 90 or 100 ng per mL of serum-free medium.

In another embodiment of the first aspect of the invention, optionally in combination with any of the embodiments provided above or below, the serum-free medium of the first aspect of the invention comprises
(a) a serum-free basal medium, and
(b) EGF; FGF protein; heparin; cell supplement, preferably B-27 supplement, preferably B-27 supplement lacking vitamin A; and VEGF.

In another embodiment of the first aspect of the invention, optionally in combination with any of the embodiments provided above or below, the serum-free medium of the first aspect of the invention comprises
(a) a serum-free basal medium, and
(b) EGF and FGF protein at a weight ratio EGF:FGF protein comprised from 1.5:1 to 8:1; heparin in an amount between 1 μg and 4 μg per mL of the culture medium; cell supplement, preferably B-27 supplement, preferably B-27 supplement lacking vitamin A, at a volume ratio vs basal medium comprised from 1:25 to 1:200; and VEGF.

In another embodiment of the first aspect of the invention, optionally in combination with any of the embodiments provided above or below, the serum-free medium of the first aspect of the invention comprises
(a) a serum-free basal medium, and
(b) EGF in an amount from 10 to 30 ng, the weight ratio EGF:FGF protein being comprised from 1.5:1 to 8:1; heparin in an amount from 1 to 100 μg per mL of culture medium; cell supplement, preferably B-27 supplement, preferably B-27 supplement lacking vitamin A, at a volume ratio vs basal medium comprised from 1:25 to 1:200; and VEGF.

In another embodiment of the first aspect of the invention, optionally in combination with any of the embodiments provided above or below, the serum-free medium of the first aspect of the invention comprises
(a) a serum-free basal medium, and
(b) EGF and FGF protein at a weight ratio 2:1, heparin in an amount between 1 μg and 4 μg per mL of culture medium; cell supplement, preferably B-27 supplement, preferably B-27 supplement lacking vitamin A, at a volume ratio vs the basal medium of 1:10 to 1:100; and VEGF.

In another embodiment of the first aspect of the invention, optionally in combination with any of the embodiments provided above or below, the serum-free medium of the first aspect of the invention comprises
(a) a serum-free basal medium, and
(b) EGF in an amount of 20 ng, EGF:FGF protein weight ratio being 2:1; heparin in an amount of 2 μg/mL per mL of culture medium; and cell supplement, preferably B-27 supplement, preferably B-27 supplement lacking vitamin A at a volume ratio vs the basal medium of 1:50; and VEGF.

In another embodiment of the first aspect of the invention, optionally in combination with any of the embodiments provided above or below, the serum-free medium of the first aspect of the invention comprises
(a) a serum-free basal medium, and
(b) EGF and FGF protein at a weight ratio EGF:FGF comprised from 1.5:1 to 8:1; heparin in an amount between 1 μg and 4 μg per mL of the culture medium; cell supplement, preferably B-27 supplement, preferably B-27 supplement lacking vitamin A, at a volume ratio vs basal medium comprised from 1:25 to 1:200; and VEGF in an amount between 1 and 10 ng per mL of serum-free medium.

In another embodiment of the first aspect of the invention, optionally in combination with any of the embodiments provided above or below, the serum-free medium of the first aspect of the invention comprises
(a) a serum-free basal medium, and
(b) EGF in an amount from 10 to 30 ng, the weight ratio EGF:FGF protein being comprised from 1.5:1 to 8:1; heparin in an amount from 1 to 100 μg per mL of culture medium; cell supplement, preferably B-27 supplement, preferably B-27 supplement lacking vitamin A, at a volume ratio vs basal medium comprised from 1:25 to 1:200; and VEGF in an amount between 1 and 10 ng per mL of serum-free medium.

In another embodiment of the first aspect of the invention, optionally in combination with any of the embodiments provided above or below, the serum-free medium of the first aspect of the invention comprises
(a) a serum-free basal medium, and
(b) EGF and FGF protein at a weight ratio 2:1; heparin in an amount between 1 μg and 4 μg per mL of culture medium; cell supplement, preferably B-27 supplement, preferably B-27 supplement lacking vitamin A, at a volume ratio vs the basal medium of 1:10 to 1:100; and VEGF in an amount between 1 and 10 ng per mL of serum-free medium.

In another embodiment of the first aspect of the invention, optionally in combination with any of the embodiments provided above or below, the serum-free medium of the first aspect of the invention comprises
(a) a serum-free basal medium, and
(b) EGF in an amount of 20 ng, EGF:FGF protein weight ratio being 2:1; heparin in an amount of 2 μg/mL per mL of culture medium; and cell supplement, preferably B-27 supplement, preferably B-27 supplement lacking vitamin A at a volume ratio vs the basal medium of 1:50; and VEGF in an amount between 1 and 10 ng per mL of serum-free medium.

In another embodiment of the first aspect of the invention, optionally in combination with any of the embodiments provided above or below, the culture medium is absent in methylcellulose. In another embodiment of the first aspect of the invention, optionally in combination with any of the embodiments provided above or below, the culture medium is absent in vitamin A. In another embodiment of the first aspect of the invention, optionally in combination with any of the embodiments provided above or below, the culture medium is absent both in vitamin A and methylcellulose.

The preparation of the culture medium of the first aspect of the invention is performed, for example, by simply mixing the different components (at least the basal medium, EGF, FGF, and VEGF, and, optionally, other components such as heparin, cell supplements, etc.).

In a second aspect, the present invention provides an in vitro serum-free process for the preparation of a cellular aggregate comprising dental stem cells and one or more differentiated endothelial cells, the process comprising culturing in suspension an isolated sample comprising dental stem cells in a serum-free cell culture medium as defined in the first aspect of the invention or any of the embodiments provided under the first aspect of the invention.

Thus, during the culturing in suspension the formation of the aggregate of stem cells occurs and, at the same time, due to presence of EGF and FGF protein at a particular ratio/amount, one or more of the stem cells which is forming the aggregate differentiates to endothelial cells.

In the present invention, the expression “serum-free process” means that the whole process to achieve the differentiation of the dental stem cell to an endothelial cell is performed in the absence of serum or serum-derived compositions. In one embodiment, the “serum-free process” means that the culture media used, from the isolation of the dental stem cells until the differentiation, do not include serum or serum-derived compositions.

In the present invention, the expression “cellular aggregate” refers to a 3D-cellular cluster which comprises starting dental stem cells as well as the endothelial cells resulting from the derivatization, among others. The cellular aggregate can include other components depending on the nature of the isolated sample used. In one embodiment, the cellular aggregate is a spheric or spheroid cellular aggregate (i.e., dentosphere).

In another embodiment of the second aspect of the invention, optionally in combination with any of the embodiments provided above or below, the “isolated sample” refers to an isolated pulp sample. In this embodiment, the isolated sample comprises, in addition to the dental stem cells, other cell types, such as endothelial cells which could be already present in blood vessels irrigating the dental pulp tissue). And, consequently, the cellular aggregate resulting from the process can include, in addition to the dental stem cells and the differentiated endothelial cells (which are derived from one another), further endothelial cells which were already present in the starting sample.

A disaggregation of the sample can be performed prior to the culturing in suspension, for example when the sample is a pulp sample.

In order to carry out the disaggregation, several processes can be used to separate the proteins from the extracellular matrix (for example, sequestering the calcium ions which allow the binding), or by proteolytically breaking said proteins (by means of the action of proteases), thus achieving a suspension of the cells present in the tissue. The methods used can generally be classified in three categories:

a) mechanical methods (for example cutting, grinding, sieving, scraping, etc.). In cell cultures, methods of scraping the plate are frequently used to pull off the adhered cells.

b) chemical methods. They generally involve the addition of solutions in which there are no divalent ions or of chelating agents for these ions. In any case, the concentrations of the ions stabilizing the binding of the proteins of the extracellular matrix and of the latter with the cell receptors is reduced.

c) enzymatic methods. Treatment of the tissue or of the cell culture with solutions of active proteases (collagenase, dispase, trypsin, elastase, papain, pronase, hyaluronidase, etc.)

In one embodiment, optionally in combination with any of the embodiments provided above or below, the reduction in the size of cellular aggregate is performed by an enzymatic method, such as by Accutase.

Alternatively, in another embodiment of the second aspect of the invention, the “isolated sample” consists of dental stem cells.

In the present invention, the term “culturing in suspension” means that the stem cells are cultured with no support or scaffold, thus remaining floating in the culture medium during their differentiation to endothelial cells.

In one embodiment of the invention, the dental stem cell is selected from the group consisting of: dental pulp stem cells (DPSCs), human exfoliated deciduous teeth (SHED), periodontal ligament stem cells (PDLSCs), dental follicle progenitor cells (DFPCs), alveolar bone-derived mesenchymal stem cells (ABMSCs), stem cells from the apical part of the human dental papilla (SOAP), tooth germ progenitor cells (TGPCs), gingival mesenchymal stem cells (GMSCs), and any combination thereof. In another embodiment of the process of the second aspect of the invention the dental stem cell is dental pulp stem cell.

DPSCs are a mesenchymal type of stem cells inside dental pulp, and were discovered in the year 2000. DPSCs have osteogenic, adipogenic and chondrogenic potential in vitro and can differentiate into dentin, in vivo and also differentiate into dentin-pulp-like complex.

Various conventional methods to isolate stem cells from dental pulp are listed below:
a) Size-sieved isolation
Enzymatic digestion of whole dental pulp tissue in solution of 3% collagenase Type I for 1 h at 37° C. is done. Through process of filtering and seeding, cells with diameter between 3 and 20 μm are obtained for further culture and amplification. Based on this approach, small-sized cell populations containing a high percent of stem cells can be isolated.
b) Stem cell colony cultivation
Enzymatic digestion of the dental pulp tissue is done to prepare single cell suspension cells of which are used for colony formation containing 50 or more cells that is further amplified for experiments.
c) Magnetic activated cell sorting (MACS)
It is an immune-magnetic method used for separation of stem cell populations based on their surface antigens (CD271, STRO-1, CD34, CD45, CD133 and c-Kit). MACS is technically simple, inexpensive and capable of handling large numbers of cells but the degree of stem cell purity is low.
d) Fluorescence activated cell sorting (FACS)
It is a convenient and efficient method that can effectively isolate stem cells from cell suspension based on cell size and fluorescence. Demerits of this technique are a requirement of expensive equipment, highly-skilled personnel, decreased viability of FACS-sorted cells and this method is not appropriate for processing bulk quantities of cells.

SHED cells were discovered in 2003. The main task of these cells seems to be the formation of mineralized tissue, which can be used to enhance orofacial bone regeneration. Advantages of banking SHED cells include: it's a simple painless technique to isolate them and being used for an autologous transplant they don't possess any risk of immune reaction or tissue rejection and hence immunosuppressive therapy is not required. SHED may also be useful for close relatives of the donor such as grandparents, parents and siblings. Apart from these, SHED banking is more economical when compared to cord blood and may be complementary to cord cell banking. The most important of all these cells are not subjected to ethical concerns as embryonic stem cells. Collection, isolation, and preservation of stem cells from human exfoliated deciduous teeth include:
Step 1: Tooth collection
Freshly-extracted tooth is transferred into vial containing hypotonic phosphate buffered saline solution (up to four teeth in one vial). Vial is then carefully sealed and placed into thermette, after which the carrier is placed into an insulated metal transport vessel. Thermette along with insulated transport vessel maintains the sample in a hypothermic state during transportation. This procedure is described as sustentation. The time from harvesting to arrival at processing storage facility should not exceed 40 h.
Step 2: Stem cell isolation
Tooth surface is cleaned by washing three times with Dulbecco's phosphate buffered saline without Ca²⁺ and Mg²⁺. Disinfection is done and again washed with PBS. Pulp tissue is isolated from the pulp chamber and is placed in sterile petri dish, washed at least three times with PBS. The tissue digestion is done with collagenase Type I and dispase for 1 h at 37° C. Isolated cells are passed through a 70 μm filter to obtain single cell suspensions. Then the cells are cultured in a MSC medium. Usually isolated colonies are visible after 24 h.
Step 3: Stem cell storage.
The approaches used for stem cell storage are: (a) Cryopreservation (b) magnetic freezing.
Cryopreservation
It is the process of preserving cells or whole tissues by cooling them to sub-zero temperatures. Cells harvested near end of log phase growth (approximately. 80-90% confluent) are best for cryopreservation. Liquid nitrogen vapour is used to preserve cells at a temperature of <−150° C. In a vial 1.5 ml of freezing medium is optimum for 1-2×10⁶ cells.
Magnetic freezing
This technology is referred to as cells alive system (CAS), which works on principle of applying a weak magnetic field to water or cell tissue which will lower the freezing point of that body by up to 6-7° C. Using CAS, Hiroshima University (first proposed this technology) claims that it can increase the cell survival rate in teeth to 83%. CAS system is a lot cheaper than cryogenics and more reliable.
The criteria of tooth eligibility for stem cells from human exfoliated deciduous teeth banking include primary incisors and canines with no pathology and at least one third of root left can be used for SHED banking. Primary molar roots are not recommended for sampling as they take longer time to resorb, which may result in an obliterated pulp chamber that contains no pulp, and thus, no stem cells. However in some cases where deciduous molars are removed early for orthodontic reasons, it may present an opportunity to use these teeth for stem cell banking.

The stem cells from apical papilla (SCAP) are the MSCs residing in the apical papilla of permanent teeth with immature roots. SCAP are capable of forming odontoblast-like cells, producing dentin in vivo, and are likely cell source of primary odontoblasts for the formation of root dentin. SCAP supports apexogenesis, which can occur in infected immature permanent teeth with periradicular periodontitis or abscess. SCAP residing in the apical papilla survive such pulp necrosis because of their proximity to the periapical tissue vasculature. Hence even after endodontic disinfection, SCAP can generate primary odontoblasts, which complete root formation under the influence of the surviving epithelial root sheath of Hertwig.

Periodontal ligament stem cells (PDLSCs) are multipotent postnatal stem cells found in the human periodontal ligament (PDLSCs). When transplanted into rodents, PDLSCs had the capacity to generate a cementum/periodontal ligament-like structure and contributed to periodontal tissue repair. These cells can also be isolated from cryopreserved periodontal ligaments while retaining their stem cell characteristics, including single-colony strain generation, cementum/periodontal-ligament-like tissue regeneration, expression of MSC surface markers, multipotential differentiation and hence providing a ready source of MSCs.

In one embodiment of the second aspect of the invention, the process comprises a step of cell expansion prior to the endothelial differentiation.

In the step of expansion the cells may be cultured in any serum-free culture medium capable of sustaining growth of the cells, such as, for example, DMEM (high or low glucose), advanced DMEM, DMEM/MCDB 201, Eagle's basal medium, Ham's F10 medium (F10), Ham's F-12 medium (F12), Iscove's modified Dulbecco's-17 medium, Mesenchymal Stem Cell Growth Medium (MSCGM), DMEM/F12, RPMI 1640, or CELL-GRO-FREE. The culture medium may be supplemented with one or more components, including, for example, beta-mercaptoethanol (BME or 2-ME); one or more growth factors (for example, vascular endothelial growth factor (VEGF), platelet-derived growth factor (PDGF), insulin-like growth factor-1 (IGF-1), leukocyte inhibitory factor (LIF) and erythropoietin (EPO)); amino acids, including L-glutamine and L-valine; and one or more antibiotic, antifungic and/or antimycotic agents to control microbial or fungal contamination (such as, for example, penicillin G. streptomycin sulphate, amphotericin B. gentamicin, fungizone and nystatin, either alone or in combination). The cells may be seeded in culture well plates, dishes of vent flasks at a density to allow cell growth.

The step of differentiation comprises suspending the dental stem cells in the culture medium defined in the first aspect of the invention or any of the embodiments provided under the first aspect of the invention. As it has been mentioned above, due to the particular composition of the medium, stem cells can effectively differentiate in the absence of any scaffold/support.

All the embodiments provided above, under the first aspect of the invention, concerning the serum-free medium, are also embodiments of the differentiation step of the process of the second aspect of the invention.

The present inventors found that when the differentiation step was performed with the culture medium of the first aspect of the invention, comprising the basal medium together with EGF, FGF2, heparin, and B27 without vitamin A, there was a near 100% efficiency in cellular aggregate generation. That is, dentospheres were obtained starting from all the samples tested.

Depending on the particular therapeutic use, it may be convenient to reduce the size of the cellular aggregate in order to avoid possible drawbacks when applied, such as the possible obstruction of the device used for its application (for example a needle).

Thus, in one embodiment of the second aspect of the invention, optionally in combination with any of the embodiments provided above or below, the process further comprises a step of reducing the size of the cellular aggregate by subjecting it to a disaggregation technique (such as those referred above).

In one embodiment, the disaggregation gives rise to a suspension just comprising smaller aggregate portions or alternatively comprising smaller aggregate portions as well as single cells (partial disaggregation).

In another embodiment of the second aspect of the invention, optionally in combination with any of the embodiments provided above or below, the process comprises a further step of isolating the cellular aggregates and/or freezing.

The skilled in the art can use any of the routine protocols for the isolation of cellular aggregates. For example, by allowing the sedimentation of the spheres and/or recovering the sediment spheroids/aggregates after a mild centrifugation (for example for 2 minutes at 120 G).

In a third aspect the present invention provides a cellular aggregate comprising dental stem cells obtainable by the process of the second aspect of the invention.

The term “obtainable” and “obtained” have the same meaning and are used interchangeably. In any case, the expression “obtainable” encompasses the term “obtained”.

In a fourth aspect, the present invention provides a culture comprising the cellular aggregate as defined in the third aspect of the invention.

In one embodiment of the fourth aspect of the invention, optionally in combination with any of the embodiments provided above or below, the culture medium is a serum-free culture medium.

In another embodiment of the fourth aspect of the invention, optionally in combination with any of the embodiments provided above or below, the culture medium is the one defined in the first aspect of the invention or in any of the embodiments provided above under the first aspect of the invention. This particular embodiment corresponds to the suspension directly obtained after the endothelial differentiation step of the process of the second aspect and corresponding embodiments. As it is shown below, the suspension obtained after the differentiation can be directly applied in a model mice to exert the beneficial effects mentioned above.

Alternatively, the cellular aggregate resulting from the process of the second aspect of the invention can be isolated from the suspension. In that case, the aggregate has to be cultured in a medium which allows cell survival without negatively compromising the bioavailability and functionality of the cellular aggregate.

In a fifth aspect the present invention provides the use of a combination comprising EGF and FGF in the differentiation of an isolated stem cell, preferably a dental stem cell, more preferably a dental pulp stem cell, to an endothelial cell.

In a sixth aspect the present invention provides the use of an endothelial cell differentiation culture medium, preferably a serum-free endothelial cell differentiation culture medium as defined in the first aspect of the invention, in differentiating a stem cell, preferably a dental stem cell, to an endothelial cell.

In one embodiment of the fifth or sixth aspect of the invention, optionally in combination with any of the embodiments provided above or below, the isolated stem cells are in the form of a suspension culture.

In one embodiment of the fifth or sixth aspect of the invention, optionally in combination with any of the embodiments provided above or below, the combination comprises the canonical human EGF protein as well as the human FGF2.

All the embodiments provided above, under the first aspect of the invention, about the basal medium, EGF, FGF, VEGF, concentrations, ratios, as well as of the inclusion of any other component such as heparin, cell supplement and other auxiliary components, and particular free-serum medium compositions are also embodiments of the fifth and sixth aspects of the invention.

In a seventh aspect the present invention provides a pharmaceutical composition comprising a therapeutically effective amount of the cellular aggregate as defined in the third aspect or of the culture as defined in the fourth aspect of the invention together with pharmaceutically acceptable excipients or carriers.

The expression “therapeutically effective amount” as used herein, refers to the amount of the active ingredient (either the aggregate or the culture) which, when administered, is sufficient to provide the therapeutic effect (neuroprotection or neovascularization). The particular dose of the active ingredient administered according to this invention will of course be determined by the particular circumstances surrounding the case, including the compound administered, the route of administration, the particular condition being treated, and the similar considerations.

The expression “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and non-human animals without excessive toxicity, irritation, allergic response, immunogenicity or other problems or complications commensurate with a reasonable benefit/risk ratio. Examples of suitable pharmaceutically acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In a tenth aspect the present invention provides the cellular aggregate as defined in the third aspect or the culture as defined in the fourth aspect or the pharmaceutical composition as defined in the seventh aspect of the invention for use as angiogenic, vasculogenic and/or neuroprotective agent.

In the present invention, the term “vasculogenic agent” means that either the cellular aggregate, the culture or the composition of the invention promotes the integration in the vascular network and/or formation of new blood vessels when there are no pre-existing ones. An “angiogenic agent” means that either the cellular aggregate, the culture or the composition of the invention promotes the formation of new blood vessels when there are pre-existing ones. A “neuroprotective agent” means that either the cellular aggregate, the culture or the composition of the invention preserve the neuronal structure and/or function.

In an eleventh aspect the present invention provides the cellular aggregate as defined in the third aspect or the culture as defined in the fourth aspect or the pharmaceutical composition as defined in the fifth aspect for use in the treatment of a disease caused by a reduction in the amount of laminin.

Laminins are high-molecular weight (˜400 to ˜900 kDa) proteins of the extracellular matrix. They are a major component of the basal lamina (one of the layers of the basement membrane), a protein network foundation for most cells and organs. The laminins are an important and biologically active part of the basal lamina, influencing cell differentiation, migration, and adhesion.

In one embodiment, the disease caused by a reduction in the amount of laminin is a neurodegenerative disease. Illustrative non-limitative examples of neurodegenerative diseases are Alzheimer's, Parkinson's or Huntington's disease.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

Methods

Cell Culture and Cell Proliferation

Human third molars were obtained from healthy donors of between 18 and 45 years of age. Tooth samples were obtained by donation after informed consent, in compliance with the 14/2007 Spanish directive for Biomedical research, and the protocol was approved by the CEISH committee of UPV/EHU.

DPSC isolation and culture were carried out as previously reported (Gronthos, S. et al 2000). Briefly, DPSCs were isolated by mechanical fracture and enzymatic digestion of the pulp tissue for 1 h at 37° C. with 3 mg/mL collagenase (17018-029, Thermo Fisher Scientific, Waltham, Mass. USA), and 4 mg/mL dispase (17105-041, Thermo Fisher Scientific, Waltham, Mass. USA). After centrifugation at 1500 rpm for 5 minutes, cells were resuspended and underwent mechanical dissociation by 18 G needles (304622, BD Microlance 3).

Then DPSCs were cultured in parallel with different types of culture media:
(i) Medium A (for comparative purposes): DMEM (Lonza 12-733, Basel, Switzerland) supplemented with 10% (v/v) of inactivated FBS (SV30160.03, Hyclone, GE Healthcare Life Sciences, Logan, Utah, USA), 2 mM L-Glutamine (G7513, Sigma, St. Louis, Mo.) and 100 U/mL penicillin+150 μg/mL streptomycin antibiotics (15140-122, Gibco).
(ii) Medium B (embodiment of the serum-free endothelial differentiation culture medium of the invention): serum-free culture medium composed of Human Neurocult NS-A basal medium (cat#05750, Stem Cell Technologies, Vancouver, Canada) with Neurocult proliferation supplement (cat#05753, Stem Cell Technologies, Vancouver, Canada) at 9:1 ratio (v/v), and supplemented with 2% (v/v) of B-27 without vitamin A, Heparin solution 2 μg/mL (cat#07980, Stem Cell Technologies, Vancouver, Canada), 20 μL of 10 μg/mL of EGF (ref#AF-100-15, Peprotech-Labomed, London, UK) to give a final concentration of 20 ng/mL and 10 μL of 10 μg/mL of FGF2 (ref#AF-100-18B, Peprotech, London, UK) to give a final concentration of 10 ng/mL in presence of antibiotics penicillin 100 U/ml and streptomycin 150 μg/ml (15140-122, Gibco).
(iii) Medium C (embodiment of the serum-free endothelial differentiation culture medium of the invention): serum-free culture medium composed of Human Neurocult NS-A basal medium (cat#05750, Stem Cell Technologies, Vancouver, Canada) supplemented with 2% (v/v) of B-27 without vitamin A, Heparin solution 2 μg/mL (cat#07980, Stem Cell Technologies, Vancouver, Canada), 0.1 μL of 100 μg/mL of VEGF-165 (ref#78159, StemCell Technologies) to give a final concentration of 10 ng/mL, 20 μL of 10 μg/mL of EGF (ref#AF-100-15, Peprotech-Labomed, London, UK) to give a final concentration of 20 ng/mL and 10 μL of 10 μg/mL of FGF2 (ref#AF-100-18B, Peprotech, London, UK) to give a final concentration of 10 ng/mL in presence of antibiotics penicillin 100 U/ml and streptomycin 150 μg/ml (15140-122, Gibco).
(iv) Medium D (embodiment of the serum-free endothelial differentiation culture medium of the invention): serum-free culture medium composed of Human Neurocult NS-A basal medium (cat#05750, Stem Cell Technologies, Vancouver, Canada) supplemented with 2% (v/v) of B-27 without vitamin A, Heparin solution 2 μg/mL (cat#07980, Stem Cell Technologies, Vancouver, Canada), 1 μL of 100 μg/mL of VEGF-165 (ref#78159, StemCell Technologies) to give a final concentration of 100 ng/mL, 20 μL of 10 μg/mL of EGF (ref#AF-100-15, Peprotech-Labomed, London, UK) to give a final concentration of 20 ng/mL and 10 μL of 10 μg/mL of FGF2 (ref#AF-100-18B, Peprotech, London, UK) to give a final concentration of 10 ng/mL in presence of antibiotics penicillin 100 U/ml and streptomycin 150 μg/ml (15140-122, Gibco).

Cells were maintained at standard conditions in a humidified 37° C. incubator containing 5% CO₂. Dentosphere cultures were then passaged every 7 days by enzymatic disaggregation with Accutase (Sigma, St. Louis, Mo.).

DPSCs were cultured for 1 month and a maximum of 4 total passages in order to avoid cell aging issues, thus resulting in the reduction of the pool of stem cells and increase of the pool of cell progenitors affecting progressively to cell viability.

The population doubling (PD) rate was determined by initial cell culture. Cells, and cellular aggregates were disaggregated, counted and passaged at day 7. At each passage cells were re-plated at the initial density and cultures were performed until passage 4. The population doubling rate was calculated using the following formula:

PD=[log₁₀(N_{f})−log₁₀(Nᵢ)]/log₁₀ (2)
(Nf is the harvested cell number and Ni is the initial plated cell number)

Cumulative population doublings (CPD) index for each passage was obtained by adding the PD of each passage to the PD of the previous passages as previously described (Pisciotta, A. et al., 2015). All cells were seeded at the density of 6×10³ cells/cm² and cultured for 1 week. Cell counting was performed after cell detachment or dissociation using an automated TC20 from Bio-Rad cell counter. The total number of cells estimation was calculated on three experimental samples for each type of culture.

### Flow Cytometry

Half-million DPSCs, grown in either DMEM 10% FBS or the serum-free proliferation media, were detached, disaggregated and then incubated with Phosphate Buffered Saline (PBS) 0.15% Bovine Serum Albumin (BSA) solution with 0.5 μg of CD31-FITC or IgG2a κ Isotype control for 1 h at 4° C. After a wash with PBS 0.15% BSA, cells were resuspended in 300 μL of PBS 0.15% BSA and analyzed using a FACS Beckman Coulter Gallios (Beckman Coulter Life Sciences, Indianapolis, United States). The data were analyzed using Flowing Software 2.5 (University of Turku, Finland).

### Animals and Cell Graft

Consanguine c57bl6 litters and Athymic Swiss^{nu/nu} (Envigo, Barcelona, Spain) were used as hosts for murine and human in vivo graft purposes. DPSCs aggregates in the active growth phase were disaggregated using Accutase (Ref: 7920, Stem Cell Technologies), washed and collected in serum-free media for its maintaining.

The disaggregation was performed in order to (a) control the number of cells administered to each animal model and (b) to reduce the possible obstruction of the needle used to administer the product to the lesion.

Two microliters containing 100,000 cells were injected (0.5 μL/min) unilaterally at the following coordinates (from Bregma): AP=−1.9, L=−1.2, and DV=−2 and −2.1. The cell transplantations were performed using a small animal stereotaxic apparatus (Kopf model 900) with a 10 μl Hamilton syringe and a 33 G needle (Hamilton, Bonaduz, Switzerland). Pre-operatory and post-operatory animal care were carried out as previously described (Jeitany M. et al., 2015).

Animals were provided with food and water ad libitum and housed in a colony isolator maintained at a constant temperature of 19-22° C. and humidity (40-50%) on a 12:12 h light/dark cycle.

The animal experiments were performed in compliance with the European Communities Council Directive of Nov. 24, 1986 (86/609/EEC) and were approved by the competent authority (Administracion Foral de Bizkaia). Immunostaining of Brain Sections and Cell Culture

Animals were deeply anesthetized with Avertin 2.5% following manufacturer's instructions, transcardially perfused with a 4% paraformaldehyde solution in 0.1 M sodium phosphate, pH 7.2, and processed as previously described (Pineda J. R. et al., 2013).

In order to detect grafted genetically unmodified human DPSCs on mice brain, specific antibodies targeted to human Nestin (MAB1259, 1:200 R&D systems), and human CD31 (BBA7, 1:200 R&D systems) were used. Immunostaining of brain vasculature was performed using CD31 (550247, 1:300 BD Pharmingen), laminin (L9393, 1:200 Sigma, St. Louis, Mo.), and VEGF (ABS82-AF647, 1:200 Sigma, St. Louis, Mo.) antibodies, and immunostaining of macrophages/microglia with CD68 antibody (MCA-1957, 1:400 BioRad).

For cell culture DPSCs, either in the form of dissociated cells or aggregates, were seeded into laminin-treated coverslips (L2020, Sigma, St. Louis, Mo.) as previously described (Silvestre D. C. et al., 2011).

After three days or one week, they were fixed by incubation with 4% Paraformaldehyde (PFA) for 10 minutes at room temperature and permeabilized by incubation in PBS 0.1% Triton X-100. Then, they were incubated overnight at 4° C. with primary antibodies at the following dilutions with PBS 0.1% Tween-20:, CD31 (550247, 1:300 BD Pharmingen, San Jose, Calif., USA), VEGF (ABS82-AF647, 1:200 Sigma, St. Louis, Mo.), laminin (L9393, 1:200 Sigma, St. Louis, Mo.).

For both tissue sections and cell culture, secondary antibodies conjugated to Alexa 488, 568 and 647 Donkey anti-mouse, anti-rabbit or anti-goat were incubated for 2 h and 30 min respectively at room temperature diluted with PBS 0.1% Tween-20. Preparations were counterstained with 4′,6-diamidino-2-phenylindole (DAPI) and images were captured using a Leica SP8 confocal microscope at 40× magnification.

Conventional PCR and Quantitative Real-Time PCR (QPCR)

RNA extraction from cell pellets, reverse transcription and QPCR were performed as previously described (Uribe-Etxebarria V. et al., 2017). The molecular weights of the amplification products were checked by electrophoresis in a 2% agarose gel. All reactions were performed in triplicate and the relative expression of each gene was calculated using the standard 2-ΔΔCt method (Livak K. J. et al., “Analysis of relative gene expression data using real-time quantitative PCR and the 2(-Delta Delta C(T)) Method”, 2001, Methods, 25, 402-408). Primer pairs used were obtained through the Primer-Blast method (Primer Bank) and they are listed in Table 1.

TABLE 1

| | | | Annealing | Amplicon |
|---|---|---|---|---|
| Primers | | Sequence 5′-3′ | (C °) | (bp) |
| β-actin | Upstream | GTTGTCGACGACGAGCG | 58.5 | 93 |
| | | (SEQ ID NO: 1) | | |
| | Downstream | GCACAGAGCCTCGCCTT | 59.7 | |
| | | (SEQ ID NO: 2) | | |
| Gapdh | Upstream | CTTTTGCGTCGCCAG | 60.3 | 131 |
| | | (SEQ ID NO: 3) | | |
| | Downstream | TTGATGGCAACAATATCCAC | 60.8 | |
| | | (SEQ ID NO: 4) | | |
| CD31 | Upstream | AGATACTCTAGAACGGAAGG | 53.01 | 120 |
| (PECAM-1) | | (SEQ ID NO: 5) | | |
| | Downstream | CAGAGGTCTTGAAATACAGG | 53.03 | |
| | | (SEQ ID NO: 6) | | |
| VEGFR2 | Upstream | GTACATAGTTGTCGTTGTAGG | 53.84 | 132 |
| | | (SEQ ID NO: 7) | | |
| | Downstream | TCAATCCCCACATTTAGTTC | 52.84 | |
| | | (SEQ ID NO: 8) | | |
| VegfA | Upstream | GACCAAAGAAAGATAGAGCAAG | 54.84 | 105 |
| | | (SEQ ID NO: 9) | | |
| | Downstream | ATACGCTCCAGGACTTATAC | 53.77 | |
| | | (SEQ ID NO: 10) | | |
| Bdnf | Upstream | TTACAAAGCTGCTAAAGTGG | 53.82 | 82 |
| | | (SEQ ID NO: 11) | | |
| | Downstream | GAACTGAGATTAGATGGCTTC | 53.82 | |
| | | (SEQ ID NO: 12) | | |

Western Blot

DPSCs either grown using medium A or B were counted and resuspended in a ratio of 20,000 cells/μL of Radioimmunoprecipitation assay (RIPA) lysis buffer (R0278, Sigma, St. Louis, Mo.) supplemented with protease (11873580001; Roche) and phosphatase inhibitors, containing a mixture of sodium fluoride, sodium orthovanadate, sodium pyrophosphate and β-glycerophosphate (Ref.: 78420, Thermo Scientific) to ensure the same cellular concentration for the different type of cells and culture media.

From this, thirty micrograms of total protein were diluted in RIPA buffer supplemented with LDS Sample Buffer (NP0007; Invitrogen by Life technologies). Electroblot was performed as previously described (Jeitany M. et al., 2015). Phosphorylated-STAT3, total STAT3, phosphorylated-ERK and total ERK antibodies were used to detect common angiogenic signalling pathways (1:1000, #9131, 1:1000, #4904S, #4370 and #4695 respectively, Cell Signalling Technologies). Beta-actin (1:1000, A5441, Sigma St. Louis, Mo.), GAPDH (1:5000, G8795, Sigma Aldrich) and Ponceau staining (P7170-1L, Sigma St. Louis, Mo.) were used as loading control to detect protein in the charged lanes.

For cell treatment to block phospho-STAT3 signalling induced by the serum-free culture media, the inventors used Stattic, an irreversible STAT3 activation inhibitor (Zhang Q, et al., 2015) that effectively blocks STAT3 signalization without affect to cell viability at doses of 1 to 2.5 μM for 72 h in culture (Li CH et al., 2018). Thus, cells were fixed and immunostained for Anti-STAT3 (phospho Y705) antibody (1:500, ab76315, Abcam) and human-CD31 (1:200, F8402, Sigma St. Louis, Mo.).

Statistical Analysis

Comparisons between multiple groups were made using Kruskal-Wallis followed by Dunn's post hoc test. Comparisons between only two groups were made using U-Mann Whitney test or Student's t test. p<0.05 was considered as statistically significant. Results were presented as mean±SD or SEM. The number of independent experiments is shown in the respective section.

Study Approval

The animal experiments were performed in compliance with the European Communities Council Directive of Nov. 24, 1986 (86/609/EEC) and were approved by the competent authority (Administración Foral de Bizkaia).

Results

Characterization of DPSC Growth Dynamics Using the Serum-Free Media Comprising EGF and FGF2

The first aim was to evaluate whether the serum-free proliferation medium comprising EGF and FGF2, would be so permissive for the growth of DPSCs.

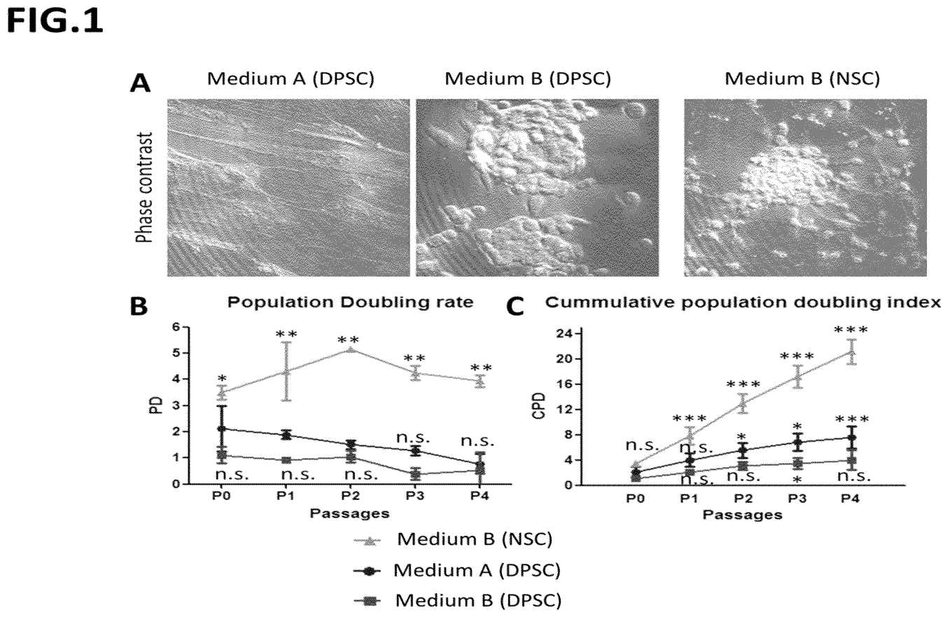

For this purpose, a comparison was made between the DPSC culture grown with the standard media DMEM/FBS (medium A) and the serum-free proliferation media (medium B, FIGS. 1(A), 1(B)). Interestingly it was found that DPSCs cultured with medium B maintained the expression of the brain-derived neurothropic factor, a neurotrophin involved in neurogenesis and neuron survival.

DPSCs Grown Using a Serum-Free Medium B Increased CD31 (Endothelial Marker) Expression

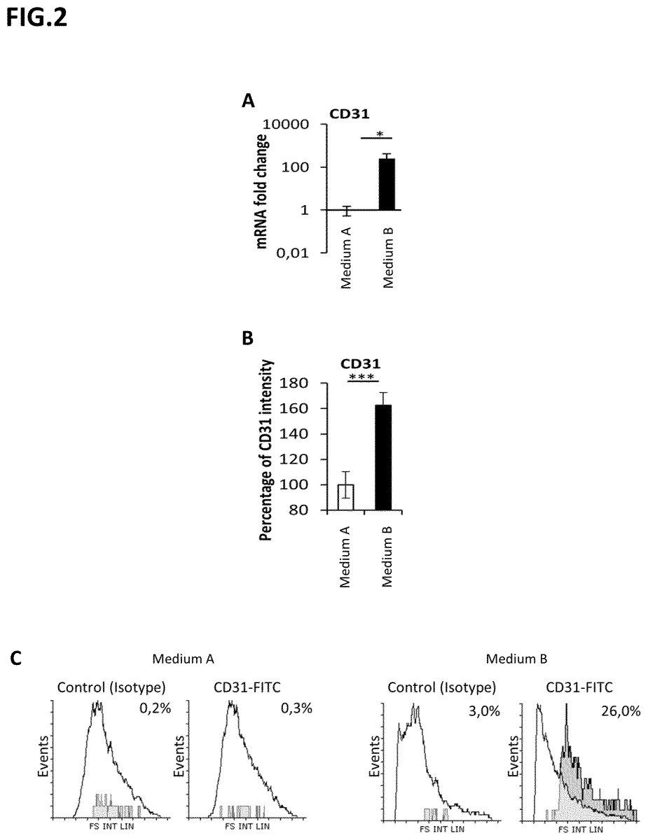

Q-PCR of CD31 mRNA expression in DPSCs cultured with serum-free medium B showed an increase up to several orders of magnitude (249,57±172,2 fold-increase of expression) with respect to cultures with medium A (p=0.0286; Mann Whitney test, FIG. 2(A)).

To confirm this result, an immunofluorescence analysis was performed in a next step for the endothelial markers CD31 and VEGF on laminin-coated slides in both DPSC grown in medium A or B, and count the percentage of CD31 intensity per cell (FIG. 2(B)).

DPSCs cultured without laminin as floating aggregates were disaggregated and thereafter a flow cytometry analysis was run by labelling the cells either with CD31-FITC or the IgG control isotype (FIG. 2(C)).

Surprisingly, it was found an increase of up to 26% of CD31-FITC positive cell population in DPSCs when these cells were grown with medium B. In contrast, the flow cytometry analysis only gave a residual value of 0.3% of CD31-positive DPSCs, when these cells were grown in medium A.

In conclusion, these results demonstrate that a serum-free medium comprising EGF and FGF2 is able to increase the expression of the CD31 endothelial cell marker in DPSC cultures.

And, consequently, this means that the cellular aggregate resulting from the culture in suspension of DPSCs in a serum-free culture medium comprising EGF and FGF2 substantially allows for an enrichment in endothelial precursor cells when compared with the result achieved with a serum-containing medium.

DPSCs Grown in Serum-Free Medium B Expressed the VEGFR2 Receptor and Showed an Increased ERK and STAT3 Pathway Activity

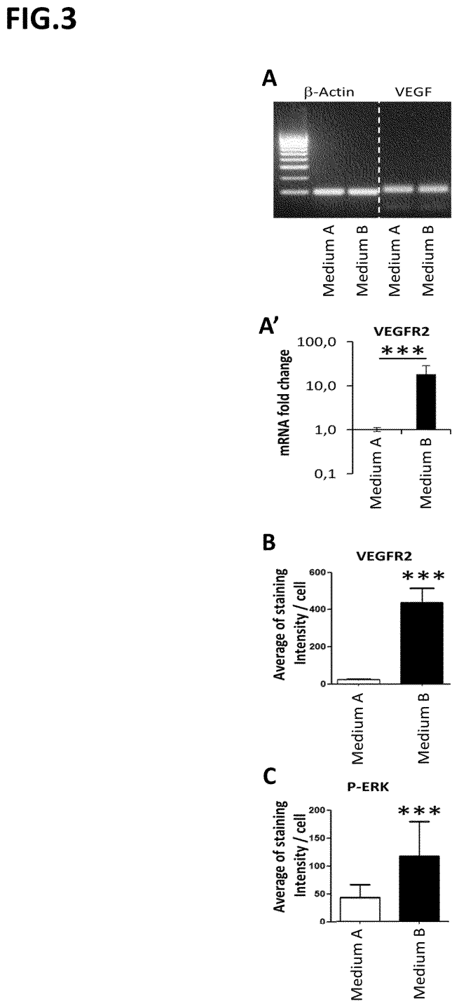

PCR detection showing a generalized VEGF expression was found in DPSCs for both culture conditions (FIG. 3(A)).

Because DPSCs grown with serum-free medium B expressed endothelial markers VEGF and CD31, it was decided to test the presence of VEGFR2 receptor at mRNA (FIG. 3(A′)) and protein level (FIG. 3(B)) by Q-PCR and immunofluorescence. DPSCs cultured with medium B showed 17.9±10.2 fold-increase of VEGFR2 mRNA with respect to cultures with medium A and a corresponding increase of VEGFR2 immunostaining.

Phosphorylation of ERK1/2 protein had been previously reported to be associated to endothelial cell differentiation of SHED. In agreement, a specific pERK staining on medium A DPSC cultures could only be detected on mitotic cells. In contrast, DPSCs cultured with serum-free medium B clearly showed an increased pERK cytoplasmic staining (FIG. 3(C)). No staining at all was found in controls without primary antibody.

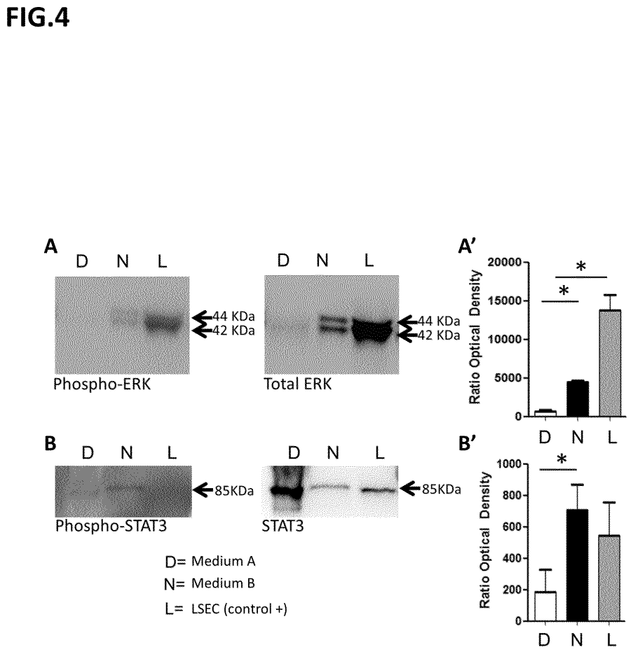

Phospho-ERK and total ERK were quantified by Western Blot, liver sinusoidal endothelial cells (LSEC) were used as positive control of mature endothelial cells. Free-floating DPSCs aggregates cultured with serum-free medium B increased ERK signalling with respect to standard medium A conditions (p=0.04 (medium A vs serum-free medium B) and p=0.0273 (medium A vs LSEC)); one-tailed Kruskal-Wallis test. FIG. 4(A-B)). STAT3 signalling in DPSCs could be abolished in the presence of inhibitors of STAT3 signalling (Stattic; at a concentration between 1 and 2.5 μM) (data not shown).

In conclusion, DPSCs grown using the serum-free medium comprising EGF and FGF2 increased the activity of the ERK and STAT3 signalling pathway, which had been already described to be essential to differentiate into endothelial cells.

DPSCs Grafted In Vivo Migrated and Integrated into Brain Vasculature

The Athymic nude mice model was chosen because it had been previously employed successfully to perform intracranial grafts of human periodontal ligament-derived cells.

Ten thousand human DPSC cells from partially disaggregated dentospheres were grafted as previously described (Pineda J. R. et al., 2013) varying the stereotaxic coordinates to place cells intrahippocampally. Cell integration in brain tissue was assessed after 30 days as previously described (Pineda J. R. et al., 2013) to better assess the long-term viability of grafted DPSC-derived cells.

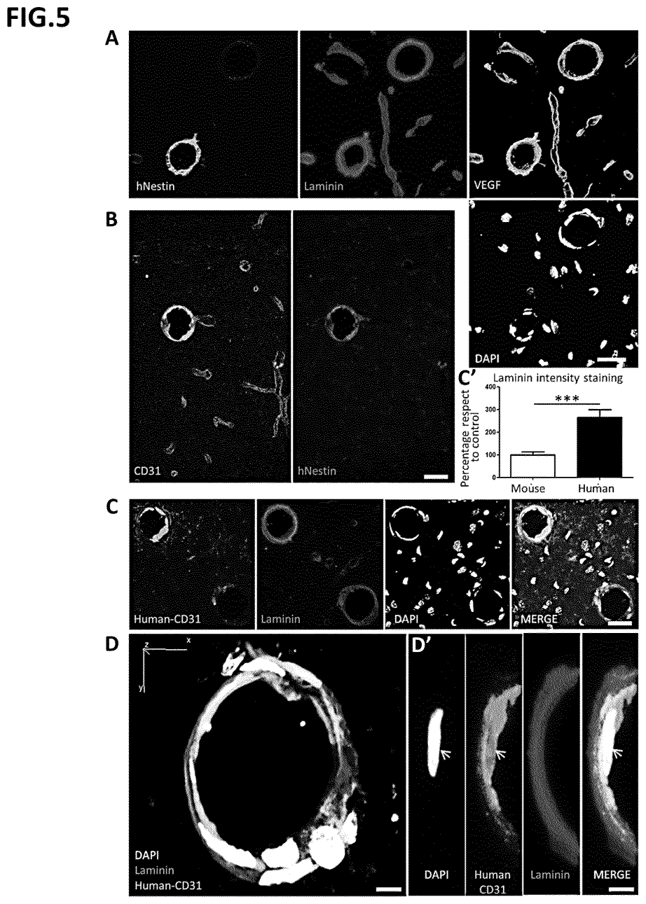

Grafted cells were located within blood vessels, which were labelled by Laminin and VEGF staining (FIG. 5(A)). Close detail allowed to determine an increased staining of laminin and VEGF in blood vessels containing human cells, with respect to blood vessels only containing the natural murine vasculature (FIG. 5(A)).

Interestingly, some of the blood vessels, including medium size blood vessels such as arterioles and venules determined by CD31 staining, appeared to be mostly generated by human DPSC-derived cells (hNestin staining), showing a very uniform and homogeneous labelling in coronal sections (FIG. 5(B)).

To further confirm this result, an immunofluorescence against CD31, using an antibody that recognized both human and murine epitopes was performed. This labelling revealed the whole murine brain vasculature, and it was found a very high level of colocalization between vascular endothelial CD31-positive cells, and human Nestin-positive cells, showing that the endothelial cells forming the inner lining of the blood vessel had primarily a human origin (FIG. 5(B)).

To further validate the presence of human endothelial cells derived from DPSC grafts, serial sections were labelled with a human-specific anti-CD31 antibody. Again, it was corroborated the presence of human CD31 positive cells integrated into the host brain vasculature (positive for Laminin staining) of Athymic nude mice at one-month post graft (FIG. 5(C)).

Remarkably, with the detection of human cells by specific CD31 antibodies, all the vasculature that contained human cells expressed higher amounts of laminin staining. This was quantified by choosing blood vessels of the same calibre, and normalizing the level of intensity of murine vasculature to 100±12%. Thus, it was determined that the presence of human DPSC-derived cells within the blood vessel raised the laminin-labelling intensity to 266±33% (p<0.0001 Student's t test, FIG. 5(C′)).

Detailed 3D-reconstruction from a 10 μm thick cryostat slice showed human-CD31 endothelial cells in the inner wall of a blood-vessel decorated with the outer laminin staining (FIG. 5(D). Close detail of a human-CD31 positive cell by separated staining showed the placement of each staining and the characteristic elongated nuclear morphology of endothelial cells (Arrow, FIG. 5(D′)).

Altogether, the experimental data provided herein allow concluding that human DPSCs, grown with serum-free proliferation medium comprising EGF+FGF2, were able to survive for one-month and functionally integrate within the brain vasculature of Athymic nude mice, expressing markers of endothelial cells. Apart for specific human-CD31 staining in the inner part of the blood vessels, complementary immunofluorescence showed specific staining against human-nestin on the outer mural part of the vessels, confirming the interpretation that the grafted heterogeneous population of human cells can induce and/or give rise to the different components of the blood vessels.

DPSCs Grown Using a Serum-Free Medium C and D Generated Dentospheres and CD31 (Endothelial Marker) Positive Cells.

The aim was to test the generation of dentospheres and CD31 positive cells replacing medium B by media C and D, which are characterized by including VEGF at low dose (medium C) or high dose (medium D).

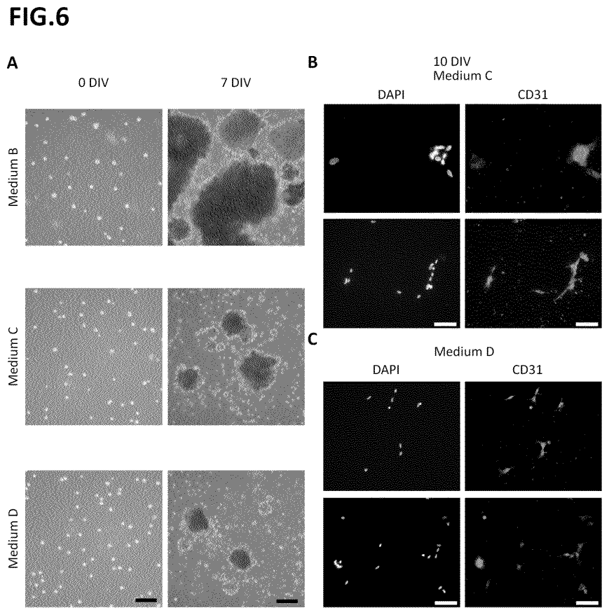

For this purpose, a comparison was made between the DPSC culture grown with the serum-free proliferation media containing proliferation supplement (medium B, FIG. 6(A)) and the serum-free proliferation media containing low dose (medium C, FIG. 6(A-B)) or high dose (medium C, FIG. 6(A-C)) of VEGF. Interestingly it was found that DPSCs cultured with medium B, C or D maintained the capacity to generate dentospheres (FIG. 6(A)).

Immunofluorescence analysis was performed in a next step after dentosphere dissociation for the endothelial marker CD31 on laminin-coated slides in both DPSC grown in medium C or D obtaining a very high proportion of CD31 endothelial cells.

In fact, comparing the data from FIG. 6 (B) and (C), supporting the effect of media C (EGF+FGF+VEGF) and D (EGF+FGF+VEGF), with those of FIG. 2 (C), supporting the effect of medium B (EGF+FGF) one can conclude that the inclusion of VEGF in the medium B allows the almost complete differentiation to endothelial cells. This means that the surprising differentiation effect provided by a free-serum culture medium comprising EGF and FGF2 can be even improved by adding VEGF.

These results open a window to use dental stem cells as a safe source to generate endothelial cells which integrate safely into the vasculature of the target tissue, providing a promising cellular therapy.

DPSCs, obtained as described in the previous example, were cultured in DMEM+FBS (DMEM (Lonza 12-733, Basel, Switzerland) supplemented with 10% (v/v) of inactivated FBS (SV30160.03, Hyclone, GE Healthcare Life Sciences, Logan, Utah, USA), 2 mM L-Glutamine (G7513, Sigma, St. Louis, Mo.) and 100 U/mL penicillin+150 μg/mL streptomycin antibiotics (15140-122, Gibco)) without laminin coating neither heparin or any supplements. Under these conditions, cells are able to survive without need of growth factors as adherent monolayer. Culture media was maintained for 7 days in parallel with the presence of 20 or 10 mL of 10 mg/mL EGF (ref#AF-100-15, Peprotech-Labomed, London, UK) to give a final concentration of 20 or 10 ng/mL and 10 mL of 10 mg/mL of FGF2 (ref#AF-100-18B, Peprotech, London, UK) to give a final concentration of 10 ng/mL.

Evidence Supporting the Differentiation Effect of EGF and FGF

Different conditions were combined as follows:

I.—DMEM+FBS (this is the control “medium A”)

II.—DMEM+FBS+FGF (New “medium E”; without EGF, only in presence of FGF)

III.—DMEM+FBS+EGF+y FGF+ (New “medium F”; equal amount of EGF and FGF (long/mL))

IV.—DMEM+FBS+EGF++y FGF+ (New “medium G”; excess of EGF (20 ng/mL) respect to FGF (10 ng/mL))

Using this four new media conditions, the effect of each one on CD31 cell expression were tested. For this purpose, 5,000 cells were seeded into glass coverslips for 7 days. Cells were immediately fixed with 4% Paraformaldehyde (PFA) for 10 minutes at room temperature and then permeabilized by incubation in PBS 0.1% Triton X-100. Coverslips were incubated overnight at 4° C. with the primary antibody CD31 (ref. ab76533, 1:200, Abcam) with PBS 0.1% Tween-20. Secondary antibody conjugated to Alexa 568 Donkey anti-rabbit was incubated for 2 h at room temperature diluted with PBS 0.1% Tween-20. Preparations were counterstained with 4′,6-diamidino-2-phenylindole (DAPI) and images were captured using a Leica SP8 confocal microscope at 40× magnification. Five aleatory regions were captured per slice in two independent experiments and data was expressed as percentage of CD31 positive cells respect total (dapi) counted cells.

The results obtained were:

I.—DMEM+FBS (this is the control “medium A”)

II.—DMEM+FBS+EGF++y FGF+ (New “medium G”; excess of EGF (20 ng/mL) respect to FGF (long/mL))

Medium A (basal culture medium): 4.5±3.8% (n=874 counted cells);

Medium G (invention): 24.9±10.0% (n=588 counted cells).

Comparation between Medium A and Medium G was statistically significant p=0.012 Mann-Whitney test) clearly showing an increase of CD31 positive cells of at least a 2-fold. This means

### REFERENCES CITED IN THE APPLICATION

Brewer G J., et al., “Survival and growth of hippocampal neurons in defined medium at low density: advantages of a sandwich culture technique or low oxygen”, 1989, Brain Res., 494(1), 65-74;

Chen Y. et al., “NS21: Re-defined and Modified Supplement B27 for Neuronal Cultures”, 2008, J. Neurosci. Methods, 171(2), 239-247;

Gronthos, S. et al., “Postnatal human dental pulp stem cells (DPSCs) in vitro and in vivo”, 2000, Proc. Natl. Acad. Sci., 97, 13625-13630;

Jeitany M. et al., “A preclinical mouse model of glioma with an alternative mechanism of telomere maintenance (ALT)”, 2015, Int. J. Cancer, 136, 1546-1558;

Li C. H. et al., “Stattic inhibits RANKL-mediated osteoclastogenesis by suppressing activation of STAT3 and NF-κB pathways”, 2018, Int. Immunopharmacol., 58, 136-144;

Pineda J. R. et al. “Vascular-derived TGF-beta increases in the stem cell niche and perturbs neurogenesis during aging and following irradiation in the adult mouse brain”, 2013, EMBO Mol. Med., 5, 548-562;

Pisciotta, A. et al., “Human dental pulp stem cells (hDPSCs): isolation, enrichment and comparative differentiation of two sub-populations”, 2015, BMC. Dev. Biol., 15, p. 14;

Romijn H. J., “Development and advantages of serum-free, chemically defined nutrient media for culturing of nerve tissue”, 1988, Biol. Cell., 63(3),263-268;

Romijn H. J. et al., “Towards an improved serum-free, chemically defined medium for long-term culturing of cerebral cortex tissue”, 1984, Neurosci. Biobehav. Rev., 8(3), 301-334.

Silvestre D. C. et al., “Alternative lengthening of telomeres in human glioma stem cells”, 2011, Stem Cells, 29, 440-451;

Uribe-Etxebarria V. et al., “Notch/Wnt cross-signalling regulates stemness of dental pulp stem cells through expression of neural crest and core pluripotency factors”, 2017, Eur Cell Mater, 34, 249-270; and

Zhang Q. et al., “STAT3 inhibitor stattic enhances radiosensitivity in esophageal squamous cell carcinoma”, 2015, Tumour Biol., 36, 2135-2142.

1. A culture medium comprising (a) a serum-free basal culture medium and (b) an endothelial cell differentiation combination of VEGF, EGF and a FGF protein, wherein the amount of EGF is higher than the amount of FGF protein.

2. The culture medium of claim 1, which further comprises one or both of heparin and a cell supplement.

3. The culture medium of claim 1, wherein it comprises VEGF, EGF, the FGF protein, heparin and the cell supplement.

4. The culture medium of claim 3, wherein:
(a) the weight ratio between EGF and the FGF protein is equal to or higher than 1.5;
(b) the VEGF is at a weight ratio from 0.1 to an excess with respect to FGF;
(c) the heparin is at a weight ratio excess with respect to EGF; and
(d) the volume ratio between the basal medium and the cell supplement is in the range from 1:25 to 1:200.

5. An in vitro serum-free process for the preparation of a cellular aggregate comprising dental stem cells, and one or more differentiated endothelial cells, the process comprising culturing in suspension an isolated sample comprising dental stem cells in a serum-free cell culture medium which is selected from:
(a) a serum-free culture medium comprising a basal culture medium, and an endothelial cell differentiation combination of EGF and a FGF protein, wherein the amount of EGF is higher than the amount of FGF protein;
(b) a serum-free culture medium comprising a basal culture medium, EGF, FGF, heparin and a cell supplement, wherein the amount of EGF is higher than the amount of FGF protein; and
(c) a serum-free culture medium the serum-free culture medium comprises:
(i) a serum-free basal culture medium and
(ii) an endothelial cell differentiation combination of VEGF, EGF and a FGF protein, wherein the amount of EGF is higher than the amount of FGF protein.

6. The in vitro process of claim 5, wherein the dental stem cell is dental pulp stem cell.

7. A cellular aggregate comprising dental stem cells and differentiated endothelial cells which is obtainable by the process of claim 5.

8. A culture comprising the cellular aggregate of claim 7.

9. A pharmaceutical composition comprising a therapeutically effective amount of the cellular aggregate of claim 7 or of a culture comprising the cellular aggregate of claim 7 together with pharmaceutically acceptable excipients or carriers.

10. A method for differentiating an isolated dental stem cell, to an endothelial cell, the method comprising culturing the dental stem cell in a serum-free culture medium comprising a combination comprising EGF and FGF protein, wherein the amount of EGF is higher than the amount of FGF protein.

11. The method of claim 10, wherein the combination further comprises VEGF.

12. The method of claim 10, wherein the culture medium is selected from:
(a) a serum-free culture medium comprising a basal culture medium, and an endothelial cell differentiation combination of EGF and a FGF protein, wherein the amount of EGF is higher than the amount of FGF protein;
(b) a serum-free culture medium comprising a basal culture medium, EGF, FGF, heparin, and a cell supplement, wherein the amount of EGF is higher than the amount of FGF protein; and
(c) a serum-free culture medium comprising:
(i) a serum-free basal culture medium and
(ii) an endothelial cell differentiation combination of VEGF, EGF and a FGF protein, wherein the amount of EGF is higher than the amount of FGF protein.

13. (canceled)

14. (canceled)

15. (canceled)

16. (canceled)

17. A method for treating a disease caused by a reduction in the amount of laminin, the method comprising administering an effective amount of a cellular aggregate of claim 7, or of a culture comprising the cellular aggregate of claim 7, or of a pharmaceutical composition comprising the cellular aggregate of claim 7, and pharmaceutically acceptable excipients or carriers, to a subject in need thereof.

18. A method of tissue regeneration, comprising administering a therapeutically effective amount of a cellular aggregate of claim 7, or of a culture comprising the cellular aggregate of claim 7, or of a pharmaceutical composition comprising the cellular aggregate of claim 7, and pharmaceutically acceptable excipients or carriers, to a subject in need thereof.

19. A method of promoting angiogenesis, comprising administering a therapeutically effective amount of a cellular aggregate of claim 7, or of a culture comprising the cellular aggregate of claim 7, or of a pharmaceutical composition comprising the cellular aggregate of claim 7, and pharmaceutically acceptable excipients or carriers, to a subject in need thereof.

20. A method of promoting vasculogenesis, comprising administering a therapeutically effective amount of a cellular aggregate of claim 7, or of a culture comprising the cellular aggregate of claim 7, or of a pharmaceutical composition comprising the cellular aggregate of claim 7, and pharmaceutically acceptable excipients or carriers, to a subject in need thereof.

21. A method of neuroprotection, comprising administering a therapeutically effective amount of a cellular aggregate of claim 7, or of a culture comprising the cellular aggregate of claim 7, or of a pharmaceutical composition comprising the cellular aggregate of claim 7, and pharmaceutically acceptable excipients or carriers, to a subject in need thereof.


SEQUENCE LISTING (12 sequences)

SEQ ID NO: 1 - DNA, 17 bp, Artificial Sequence
  upstream primer for beta-actin
gttgtcgacgacgagcg

SEQ ID NO: 2 - DNA, 17 bp, Artificial Sequence
  downstream primer for beta-actin
gcacagagcctcgcctt

SEQ ID NO: 3 - DNA, 15 bp, Artificial Sequence
  upstream primer for Gapdh
cttttgcgtcgccag

SEQ ID NO: 4 - DNA, 20 bp, Artificial Sequence
  downstream primer for Gapdh
ttgatggcaacaatatccac

SEQ ID NO: 5 - DNA, 20 bp, Artificial Sequence
  upstream primer for CD31
agatactctagaacggaagg

SEQ ID NO: 6 - DNA, 20 bp, Artificial Sequence
  downstream primer for CD31
cagaggtcttgaaatacagg

SEQ ID NO: 7 - DNA, 21 bp, Artificial Sequence
  upstream primer for VEGFR2
gtacatagttgtcgttgtagg

SEQ ID NO: 8 - DNA, 20 bp, Artificial Sequence
  downstream primer for VEGFR2
tcaatccccacatttagttc

SEQ ID NO: 9 - DNA, 22 bp, Artificial Sequence
  upstream primer for VegfA
gaccaaagaaagatagagcaag

SEQ ID NO: 10 - DNA, 20 bp, Artificial Sequence
  downstream primer for VegfA
atacgctccaggacttatac

SEQ ID NO: 11 - DNA, 20 bp, Artificial Sequence
  upstream primer for Bdnf
ttacaaagctgctaaagtgg

SEQ ID NO: 12 - DNA, 21 bp, Artificial Sequence
  downstream primer for Bdnf
gaactgagattagatggcttc